(19) United States
(12) Patent Application Publication
(10) Pub. No.: US 2006/0035876 A1
(43) Pub. Date: Feb. 16, 2006

(54) Combination therapy of an SODm and a corticosteroid for prevention and/or treatment of inflammatory bone or joint disease

(75) Inventor: Daniela Salvemini, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: MetaPhore Pharmaceuticals, Inc.

(21) Appl. No.: 11/141,550

(22) Filed: May 31, 2005

Related U.S. Application Data
(63) Continuation of application No. 10/481,396, filed on Jun. 2, 2004.
(60) Provisional application No. 60/301,080, filed on Jun. 26, 2001.

(51) Int. Cl. A61K 31/573; A61K 31/555

(52) U.S. Cl. 514/171; 514/185

(57) ABSTRACT

The present invention relates to pharmaceutical compositions and methods of using such compositions for the treatment of inflammatory diseases of the bone and joints. The compositions comprise a catalyst for the dismutation of superoxide, which is a non-proteinaceous mimetic of superoxide dismutase, in combination with a corticosteroid. The combination is substantially more effective than either the superoxide dismutase mimetic or the corticosteroid given alone at the same dose. Treatment with the combination beneficially alters the progression of the inflammatory bone and joint disease as measured histologically in diminished bone resorption and infiltration of inflammatory cells; as measured radiographically in diminished joint erosion, and diminished bone erosion and osteophyte formation; and as measured histomorphometrically in decreased bone resorption measurements of eroded surface and/or osteoclast surface relative to bone surface, and increased bone formation measurements of osteoblast surface relative to bone surface.

### RELATED APPLICATIONS

This application is a continuation in part of U.S. application Ser. No. 10/481,396 which is a national stage application of PCT application No. PCT/US02/20476 filed Jun. 26, 2002, which in turn claims the benefit of U.S. Provisional Application No. 60/301,080, filed Jun. 26, 2001. All of the above-mentioned applications are incorporated herein by reference in their entirety.

### FIELD

The present invention relates generally to compositions and methods for the treatment of inflammatory diseases and, more particularly, to combinations of corticosteroids and superoxide dismutase catalysts which are manganese or iron complexes of substituted, unsaturated heterocyclic pentaazacyclopentadecane ligands and to methods of treating inflammatory diseases with the combinations.

### INTRODUCTION

Inflammatory disease is any disease marked by inflammation, which is a localized protective response elicited by injury or destruction of tissues. The inflammation serves to destroy, dilute, or separate both the injurious agent and the injured tissue. In the acute form, inflammation can be characterized by the classical signs of pain, heat, redness, swelling and loss of function. Inflammation occurs when, upon injury, recruited polymorphonuclear leukocytes release Reactive Oxygen Species (ROS) in oxidative bursts resulting in a complex cascade of events. Histologically, it involves a complex series of events, including dilation of arterioles, capillaries, and venules, with increased permeability and blood flow; exudation of fluids, including plasma proteins; and leukocytic migration into the inflammatory focus. One of the most prominent of inflammatory diseases is arthritis, which refers to inflammation of the joints. Other inflammatory diseases include inflammatory bowel disease, asthma, psoriasis, lupus and other autoimmune diseases. The inflammation of the inflammatory diseases may be caused by a multitude of inciting events, including radiant, mechanical, chemical, infectious, and immunological stimuli.

One of the most prominent inflammatory diseases is arthritis. Arthritis is a term that refers to a group of more than 100 diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis and rheumatoid arthritis. Rheumatoid arthritis is the most severe of these two forms in terms of pain, while osteoarthritis is the most common form. Rheumatoid arthritis is a systematic, inflammatory, autoimmune disease that commonly affects the joints, particularly those of the hands and feet. Autoimmune diseases are caused by an abnormal immune response involving either cells or antibodies directed against normal tissues. A number of strategies have been developed to suppress autoimmune diseases, most notably drugs which nonspecifically suppress the immune response. The onset of rheumatoid arthritis can occur slowly, ranging from a few weeks to a few months, or the condition can surface rapidly in an acute manner.

At the cellular level, inflammatory diseases are characterized by an accumulation of cytokines such as TNF-α; IL-1β, IL-6, IL-9, IL-11, IL-15, IL-5 and 15 several belonging to the interferon family, as well as inflammatory cells (e.g., eosinophils, neutrophils, and macrophages). Focussing on arthritis specifically, these cytokines accumulate in synovial fluid during arthritic flare-up. Many of these cytokines are released from inflammatory cells which in turn cause cell and tissue damage. Additionally, another significant characteristic of the inflammatory response associated with arthritis and other diseases like lupus is a process called autoimmunity. Autoimmunity occurs when T-cells mistake the body's own collagen cells as foreign antigens and set off a series of events to clear the erroneously perceived threat. This results in an attack of the body's own cells by its immune system. Autoimmunity is particularly associated with rheumatoid arthritis and lupus. The immune response associated with arthritic flare-up is also characterized by oxidative and nitrosative stress and polyADP-ribose synthetase (PARS) activity.

Aspirin is widely used to treat pain and to reduce inflammation in many inflammatory diseases. In addition to aspirin, non-steroidal anti-inflammatory drugs, corticosteroids, gold salts, anti-malarials and systemic immunosuppressants are widely used in moderate to advanced cases of arthritis and other inflammatory diseases. Corticosteroids are effective drugs for the treatment of arthritis, other inflammatory diseases and the pain associated with these disease and these compounds are the most potent anti-inflammatory agents previously known.

Corticosteroids can be classified as glucocorticoids and mineralocorticoids. The effects of corticosteroids are numerous and widespread. Some of these effects include: alterations in carbohydrate, protein, and lipid metabolism; maintenance of fluid and electrolyte balance; and preservation of normal function of the cardiovascular system, the immune system, the kidney, skeletal muscle, the endocrine system, and the nervous system. The mechanisms of corticosteroids are still not fully understood, but corticosteroids endow the organism with the capacity to resist stressful circumstances such as noxious stimuli and environmental changes. One of the major pharmaceutical uses for corticosteroids are as anti-inflammatory and immunosuppressive agents. The pharmacological actions of corticosteroids in different tissues and many of their physiological effects seem to be mediated by the same receptor.

For many years corticosteroids have been used for treating inflammatory conditions. Generally, prednisone, an alcohol, is used orally, and the corresponding ketone prednisolone (or methyl-prednisolone) is used for parenteral injections. These compounds are five times more effective than naturally occurring cortisone which tends to minimize toxicity problems. More-developed fluorinated derivatives of corticosteroids (e.g., triamcinolone, dexamethasone, paramethasone, and betamethasone) are three to five times more effective than non-fluorinated compounds, however, these compounds are also more toxic. Corticosteroids are the most widely used anti-inflammatory drugs for both acute and chronic inflammation. They are used orally, parenterally, and frequently, intra- and peri-articularly, i.e., injected in and around joints and joint cavities. However, the side effects associated with corticosteroid use can often be severe.

Reactive oxygen species and their metabolites have been postulated to contribute to a number of tissue pathologies. Such reactive oxygen species include the superoxide anion (O₂⁻), hydroxyl radical (OH⁻), and nitric oxide (NO⁻) as well as other species. The diseases involving reactive oxygen species can include inflammatory diseases, such as reperfusion injury (particularly for the intestine, liver, heart and brain), inflammatory bowel disease, rheumatoid arthritis, osteoarthritis, atherosclerosis, hypertension, cancer, skin disorders (e.g., psoriasis, dermatitis), organ transplant rejections, chemotherapy and radiation-induced side effects, pulmonary disorders (e.g., chronic obstructive pulmonary disease (COPD), asthma), influenza, stroke, burns, AIDS, malaria, Parkinson's disease and trauma. See, for example, Simic, M. G., et al, “Oxygen Radicals in Biology and Medicine”, Basic Life Sciences, Vol. 49, Plenum Press, New York and London, 1988; Weiss J. Cell. Biochem., 1991 Suppl. 15C, 216 Abstract C110 (1991); Petkau, A., Cancer Treat. Rev. 13, 17 (1986); McCord; J. Free Radicals Biol. Med., 2, 307 (1986); and Bannister, J. V. et al, Crit. Rev. Biochem., 22, 111 (1987). Reactive oxygen species are believed to contribute significantly to tissue injury in rheumatoid arthritis and other inflammatory diseases. See Bauerova et al., “Role of Reactive Oxygen and Nitrogen Species in Etiopathogenesis of Rheumatoid Arthritis” Gen Physiol Biophys 1999 Oct.; 18 Spec No.: 15-20.

Clinical trials and animal studies with natural, recombinant and modified superoxide dismutase have been completed or are ongoing to demonstrate the therapeutic efficacy of reducing superoxide levels in the disease states noted above. However, numerous problems have arisen with the use of the proteinaceous enzymes as potential therapeutic agents, including lack of oral activity, short half-lives in vivo, immunogenicity with nonhuman derived enzymes, and poor tissue distribution.

Thus, the need exists for effective compositions and methods for preventing and treating inflammatory disorders including rheumatoid arthritis and other inflammatory diseases associated with the overproduction of reactive oxygen species.

### SUMMARY

Accordingly, the present invention provides a method for treating inflammatory disease in a subject. The method comprises co-administering a therapeutically effective amount of a catalyst for the dismutation of superoxide in conjunction with at least one corticosteroid. The method is particularly applicable to the treatment of inflammatory diseases of the joints and bones, including rheumatoid arthritis.

Thus, in various embodiments, the present invention is directed to a method of treating an inflammatory disease of a bone and/or joint. The method comprises administering to a subject in need thereof, an effective amount of a combination of a catalyst for the dismutation of superoxide and a corticosteroid. In various aspects of this embodiment, the amount of the catalyst in the combination or the amount of the corticosteroid in the combination or both, is less than an amount that can be used to effectively treat the inflammatory disease. Thus, the amount of either or both the catalyst or the corticosteroid when administered alone, is not substantially effective in treating the disease. Nevertheless, the combination of the catalyst and the corticosteroid is substantially effective in treating the inflammatory disease.

The present invention, in various embodiments, also includes a method of effectively treating an inflammatory disease of a bone and/or joint with a reduced dose of a corticosteroid. The method comprises administering to a subject in need thereof, an effective amount of a combination of the corticosteroid and a catalyst for the dismutation of superoxide. The amount of the corticosteroid in the combination, is less than an amount that can be used to effectively treat the inflammatory disease, i.e., the amount, when administered alone is not substantially effective in treating the disease. Nevertheless, the combination of the catalyst and the corticosteroid is substantially effective in treating the inflammatory disease.

In various embodiments, the present invention includes a method of increasing the effectiveness of a corticosteroid in treating an inflammatory disease of a bone and/or joint. The method comprises administering to a subject in need thereof, an effective amount of a combination of the corticosteroid and a catalyst for the dismutation of superoxide. The effectiveness of the combination in treating the inflammatory disease is greater than that of the corticosteroid when administered alone in the same amount.

The present invention, in various embodiments, also includes a method of improving one or more measures of an inflammatory disease selected form the group consisting of histologic measures, radiographic measures, histomorphometric measures and combinations thereof. The method comprises administering to a subject an effective amount of a combination of a non-proteinaceous catalyst for dismutation of superoxide and a corticosteroid.

In various embodiments, the present invention includes a method of preventing or diminishing either or both of bone resorption and infiltration of inflammatory cells. The method comprises administering to a subject an effective amount of a combination of a non-proteinaceous catalyst for dismutation of superoxide and a corticosteroid.

The present invention, in various embodiments, can also include a method of preventing or diminishing bone erosion, osteophyte formation, joint erosion or any combination thereof. The method comprises administering to a subject an effective amount of a combination of a non-proteinaceous catalyst for dismutation of superoxide and a corticosteroid.

In various embodiments, of the present invention, the catalyst for the dismutaion of superoxide can be a non-proteinaceous catalyst represented by the formula: 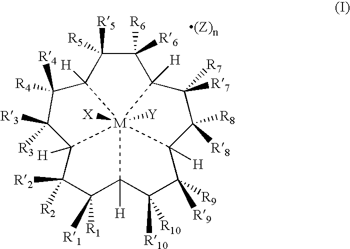
wherein

(i) one or more of R¹, R′, R², R′², R³, R′³, R⁴, R′⁴, R⁵, R′⁵, R⁶, R′⁶, R⁷, R′⁷, R⁸, R′⁸ , R⁹, R′⁹, R¹⁰ and R′¹⁰ are independently:

(ia) hydrogen; or

(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino acids, wherein R11 and R12 are independently hydrogen or alkyl; and

(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and

(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰ together with the carbon atom to which they are attached independently form a substituted or unsubstituted and saturated, partially saturated, or unsaturated cycle or heterocycle having 3 to 20 carbon atoms; and

(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(v) optionally, one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ , which is attached to a different carbon atom in the macrocyclic ligand may be bound to form a strap represented by the formula:
(CH₂)_{I}(CH₂)_{J}R(CH₂)_{K}S(CH₂)_{L}
wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vi) combinations of any of (i) through (v) above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge neutralizing anions which are derived from any monodentate or polydentate coordinating ligand and a ligand system and the corresponding anion thereof, or

X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰; and

n is an integer from 0 to 3.

### FIGURES

FIG. 1. Effect of combination therapy (dexamethasone (DEX) 0.01 mg/kg+M40403 2 mg/kg) on the onset of collagen-induced arthritis. The percentage of arthritic rats (rats showing clinical scores of arthritis are shown in panel (A). Median arthritic score during collagen-induced arthritis is shown in panel (B). Values are means±standard error of the mean (s.e.m.) of 10 animals for each group. *p<0.01 versus Control. ° p<0.01 versus CIA.

FIG. 2. Effect of combination therapy (DEX 0.01 mg/kg+M40403 2 mg/kg) on paw swelling. Values are means±s.e.m. of 10 animals for each group. *p<0.01 versus Control. ° p<0.01 versus CIA.

FIG. 3. Plasma levels of TNF-α(A) and IL-1β(B). Cytokine levels were significantly reduced in the plasma from rats which received DEX (0.1 mg/kg) or combination therapy (DEX 0.01 mg/kg+M40403 2 mg/kg). Values are means U±s.e.m. of 10 animals for each group. *p<0.01 versus sham. ° p<0.01 versus CIA.

FIG. 4. Effect of combination therapy (DEX 0.01 mg/kg+M40403 2 mg/kg) malondialdehyde (MDA) levels in plasma: MDA levels in the plasma of CII-immunized rats killed at 35 days. MDA levels were significantly increased in the plasma of the CII-immunized rats in comparison to sham rats (*p<0.01). DEX (0.1 mg/kg) or combination therapy (DEX 0.01 mg/kg+M40403 2 mg/kg) reduced the CIA increase in MDA levels. Values are means±s.e.m. of 10 rats for each group. *p<0.01 versus shamp. ° p<0.01 versus CIA.

FIG. 5. Nitrotyrosine immunostaining in the paw of a control rat (A) and the paw of a rat at 35 days of collagen-induced arthritis (B). A marked increase in Nitrotyrosine staining is evident in the paws in arthritis. There was a marked reduction in the immunostaining in the paw of rats which were treated with DEX (0.1 mg/kg) (C) or with combination therapy (DEX 0.01 mg/kg+M40403 2 mg/kg) (D). Original magnificantion: X125. Figure is representative of at least 3 experiments performed on different experimental days.

FIG. 6. Effect of combination therapy (DEX 0.01 mg/kg+M40403 2 mg/kg) on PARS activity: Staining was absent in control tissue (A). 35 days following collagen-induced arthritis, PARS immunoreactivity was present in the paw from CII-immunized rats (B). There was a marked reduction in the immunostaining in the paw of rats which were treated with DEX (0.1 mg/kg) (C), or combination therapy (DEX 0.01 mg/kg+M40403 2 mg/kg) (D) no positive staining was found. Original magnification: X125. Figure is representative of at least 3 experiments performed on different experimental days.

FIG. 7. Plasma levels of nitrite/nitrate (NOₓ). NOₓ levels were significantly reduced in the plasma from rats which received DEX (0.1 mg/kg) or combination therapy (DEX 0.01.mg/kg+M40403 2 mg/kg). Values are means±s.e.m. of 10 animals for each group. *p<0.01 versus sham. ° p<0.01 versus CIA.

FIG. 8. Inducible nitric oxide synthase (iNOS) immunostaining in the paw of a control rat (A) and the paw of a rat at 35 days of collagen-induced arthritis (B). A marked increase in iNOS staining is evident in the paws afflicted with arthritis. There was a marked reduction in the immunostaining in the paw of rats which were treated with DEX (0.1 mg/kg) (C) or a combination therapy (DEX 15 0.01 mg/kg+M40403 2 mg/kg) (D). Original magnification: X125. The figure is representative of at least 3 experiments performed on different experimental days.

FIG. 9. Effect of combination therapy (DEX 0.01 mg/kg+M40403 2 mg/kg) on COX-2 expression: Staining was absent in control tissue (A). 35 days following collagen-induced arthritis, COX-2 immunoreactivity was present in the paw from CII-immunized rats (B). In the paw of rats which received DEX (0.1 mg/kg) (C), or combination therapy (DEX 0.01 mg/kg+M40403 2 mg/kg) (D) no positive staining was found. Original magnification: X125. Figure is representative of at least 3 experiments performed on different experimental days.

FIG. 10. Effect of combination therapy (DEX 0.01 mg/kg+M40403 2 mg/kg) on body weight gain. Beginning on day 25, the collagen-challenged rats or rats treated with low doses of DEX (0.01 mg/kg) or M40403 (2 mg/kg) alone gained significantly less weight than the normal rats, and this trend continued through day 35. On the other hand, DEX at the high dose tested (0.1 mg/kg) or combination of low doses DEX and M40403 (0.01 mg/kg+2 mg/kg respectively) gained weight in a manner similar to sham animals. Values are means±s.e.m. of 10 animals for each group. *p<0.01 versus Control. ° p<0.01 versus CIA.

FIG. 11. This figure illustrates histologic features (original magnification X125) of the paws of (A) a control animal, (B) an animal having collagen-induced arthritis (CIA) elicited by immunization with bovine type II collagen (CII), (C) an arthritic animal treated with dexamethasone (0.1 mg/kg) and (D) an arthritic animal given the combination therapy of M40403 (2 mg/kg) plus dexamethasone (0.01 mg/kg).

FIG. 12. This figure illustrates the histologic damage scores as determined by 2 independent observers in animals with CIA and treated with vehicle, M40403 (2 mg/kg), dexamethasone (DEX)(0.01 mg/kg or 0.1 mg/kg) or the combination of dexamethasone (0.01 mg/kg) and M40403 (2 mg/kg). Scores are representative of at least 3 experiments performed on different experimental days. Values are the mean and SEM of 10 rats per group. *p<0.01 versus sham-treated rats; ° p<0.01 versus CII-treated rats, by Mann-Whitney U test.

FIG. 13. This figure illustrates the radiographic progression of CIA in the tibiotarsal joints of rats showing (A) no evidence of pathology in the tibiotarsal joints of normal animals, (B) bone resorption (arrow) in the hind paws from CII-immunized (35 days) rats, (C) the hind paws of animals receiving dexamethasone (0.01 mg/kg) and (D) suppression of joint pathology in animals receiving combination therapy with M40403 (2 mg/kg) and dexamethasone (0.01 mg/kg).

FIG. 14. This figure illustrates the radiographic scores as determined by 2 independent observers. Scores are representative of at least 3 experiments performed on different experimental days. Animals with CIA were treated with vehicle, M40403 (2 mg/kg), dexamethasone (DEX)(0.01 mg/kg or 0.1 mg/kg) or the combination of dexamethasone (0.01 mg/kg) and M40403 (2 mg/kg). Scores are representative of at least 3 experiments performed on different experimental days. Values are the mean and SEM of 10 rats per group. *p<0.01 versus sham-treated rats; ° p<0.01 versus CII-treated rats, by Mann-Whitney U test.

FIG. 15. This figure demonstrates the effect of combination therapy (DEX in μM+30 μM of M40401) on the LPS-stimulated TNF-α in LPS treated RAW cells.

FIG. 16. This figure demonstrates the effects of the oxidation product obtained from the reaction of dexamethasone with superoxide, tested in vitro for its ability to inhibit TNF-α production. The figure shows that the oxidation product has no activity on TNF-α.

FIG. 17. This figure demonstrates the effects of dexamethasone and FeTMPS ((5,10,15,20-tetrakis (2,4,6-trimethyl-3,5-disulfonatophenyl)-porphyrinate iron (III)) in carrageenan-induced paw edema. The results show that a low dose of FeTMPS (1 mg/kg) (note: mg/kg is also expressed as mpk) when combined with low dose of Dexamethasone (0.1 mg/kg) enhances the effects of Dexamethasone such that the combination dosage is equivalent to giving a higher dose of 3 mg/kg of Dexamethasone.

### DETAILED DESCRIPTION

The present invention, in various embodiments, includes methods and compositions for the prevention and treatment of inflammatory diseases involving administration of a combination of a corticosteroid and a non-proteinaceous catalyst for the dismutation of superoxide.

As used herein, the term “corticosteroid” refers to any of the adrenal corticosteroid hormones isolated from the adrenal cortex or produced synthetically, and derivatives thereof that are used for treatment of inflammatory diseases, such as arthritis, asthma, psoriasis, inflammatory bowel disease, lupus, and others. Corticosteroids include those that are naturally occurring, synthetic, or semi-synthetic in origin, and such compounds are characterized by the presence of a steroid nucleus of four fused rings, e.g., as found in cholesterol, dihydroxycholesterol, stigmasterol, and lanosterol structures. Corticosteroid drugs include cortisone, cortisol, hydrocortisone (11β, 17-dihydroxy, 21-(phosphonooxy)-pregn-4-ene, 3,20-dione disodium), dihydroxycortisone, dexamethasone (21-(acetyloxy)-9-fluoro-11β,17-dihydroxy-16α-methylpregna-1,4-diene-3,20-dione), and highly derivatized steroid drugs such as beconase (beclomethasone dipropionate, which is 9-chloro-11β,17,21, trihydroxy-16β-methylpregna-1,4diene-3,20-dione 17,21-dipropionate). Other examples of corticosteroids include flunisolide, prednisone, prednisolone, methylprednisolone, triamcinolone, deflazacort and betamethasone.

As used herein, the terms “reactive oxygen species” or “ROS” refers to a toxic or reactive superoxide anion (O₂⁻). The superoxide anion, as well as the nitric oxide (NO⁻) and the hydroxyl radical (OH⁻) are different types of free-radicals.

As used herein, the terms “non-peptidic catalysts for the dismutation of superoxide” or “non-proteinaceous catalysts for the dismutation of superoxide” mean a low-molecular weight catalyst for the conversion of superoxide anions into hydrogen peroxide and molecular oxygen. These catalysts commonly consist of an organic ligand and a chelated transition metal ion, preferably copper, manganese(II), manganese(III), iron(II) or iron(III). The term may include catalysts containing short-chain polypeptides (under 15 amino acids) or macrocyclic structures derived from amino acids, as the organic ligand. The term explicitly excludes a superoxide dismutase enzyme (SOD) obtained from any species.

The term “catalyst for the dismutation of superoxide” means any catalyst for the conversion of superoxide anions into hydrogen peroxide and molecular oxygen. The term explicitly includes a superoxide dismutase enzyme (SOD) obtained from any species.

The term “substituted” means that the described moiety has one or more substituents comprising at least 1 carbon or heteroatom, and further comprising 0 to 22 carbon atoms, more preferably from 1 to 15 carbon atoms, and comprising 0 to 22 heteroatoms, more preferably from 0 to 15 heteroatoms. As used herein, “heteroatom” refers to those atoms that are neither carbon nor hydrogen bound to carbon and such atoms are selected from the group consisting of: O, S, N, P, Si, B, F, CI, Br, or I. These atoms may be arranged in a number of configurations, creating substituent groups which are unsaturated, saturated, or aromatic. Examples of such substituents include branched or unbranched alkyl, alkenyl, or alkynyl, cyclic, heterocyclic, aryl, heteroaryl, allyl, polycycloalkyl, polycycloaryl, polycycloheteroaryl, imines, aminoalkyl, hydroxyalkyl, hydroxyl, phenol, amine oxides, thioalkyl, carboalkoxyalkyl, carboxylic acids and their derivatives, keto, ether, aldehyde, amine, amide, nitrite, halo, thiol, sulfoxide, sulfone, sulfonic acid, sulfide, disulfide, phosphoric acid, phosphinic acid, acrylic acid, sulphonamides, amino acids, peptides, proteins, carbohydrates, nucleic acids, fatty acids, lipids, nitro, hydroxylamines, hydroxamic acids, thiocarbonyls, thiocarbonyls, borates, boranes, boraza, silyl, silaza, siloxy, and combinations thereof.

The term “alkenyl”, alone or in combination, means an alkyl substituent having one or more double bonds. Examples of such alkenyl substituents include, but are not limited to, ethenyl, propenyl, 1-butenyl, cis-2-butenyl, trans-2-butenyl, iso-butylenyl, cis-2-pentenyl, trans-2-pentenyl, 3-methyl-1-butenyl, 2,3-dimethyl-2-butenyl, 1-pentenyl, 1-hexenyl, 1-octenyl, decenyl, dodecenyl, tetradecenyl, hexadecenyl, cis- and trans-9-octadecenyl, 1,3-pentadienyl, 2,4-pentadienyl, 2,3-pentadienyl, 1,3-hexadienyl, 2,4-hexadienyl, 5,8,11,14-eicosatetraenyl, and 9,12,15-octadecatrienyl.

The term “alkyl”, alone or in combination, means a straight-chain or branched-chain alkyl substituent containing from 1 to about 22 carbon atoms, prefarably from about 1 to about 18 carbon atoms, and most preferably from about 1 to about 12 carbon atoms. Examples of such substituents include, but are not limited to, methyl, ethyl, n-propyl, isopropyl, n-butyl, isobutyl, sec-butyl, tert-butyl, pentyl, iso-amyl, hexyl, octyl, nonyl, decyl, dodecyl, tetradecyl, hexadecyl, octadecyl and eicosyl.

The terms “alkylcycloalkyl” and “alkenylcycloalkyl” mean a cycloalkyl substituent as defined above which is substituted by an alkyl or alkenyl substituent as defined above. Examples of alkylcycloalkyl and alkenylcycloalkyl substituents include, but are not limited to, 2-ethylcyclobutyl, 1-methylcyclopentyl, 1-hexylcyclopentyl, 1-methylcyclohexyl, 1-(9-octadecenyl)cyclopentyl and 1-(9-octadecenyl)cyclohexyl.

The terms “alkylcycloalkenyl” and “alkenylcycloalkenyl” means a cycloalkenyl substituent as defined above which is substituted by an alkyl or alkenyl substituent as defined above. Examples of alkylcycloalkenyl and alkenylcycloalkenyl substituents include, but are not limited to, 1-methyl-2-cyclopentyl, 1-hexyl-2-cyclopentenyl, 1-ethyl-2-cyclohexenyl, 1-butyl-2-cyclohexenyl, 1-(9-octadecenyl)-2-cyclohexenyl and 1-(2-pentenyl)-2-cyclohexenyl.

The term “alkynyl”, alone or in combination, means an alkyl substituent having one or more triple bonds. Examples of such alkynyl groups include, but are not limited to, ethynyl, propynyl (propargyl), 1-butynyl, 1-octynyl, 9-octadecynyl, 1,3-pentadiynyl, 2,4-pentadiynyl, 1,3-hexadiynyl, and 2,4-hexadiynyl.

The term “aralkyl”, alone or in combination, means an alkyl or cycloalkyl substituent as defined above in which one hydrogen atom is replaced by an aryl substituent as defined above, such as benzyl, 2-phenylethyl, and the like.

The term “aryl”, alone or in combination, means a phenyl or naphthyl substituent which optionally carries one or more substituents selected from alkyl, cycloalkyl, cycloalkenyl, aryl, heterocycle, alkoxyaryl, alkaryl, alkoxy, halogen, hydroxy, amine, cyano, nitro, alkylthio, phenoxy, ether, trifluoromethyl and the like, such as phenyl, p-tolyl, 4-methoxyphenyl, 4-(tert-butoxy)phenyl, 4-fluorophenyl, 4-chlorophenyl, 4-hydroxyphenyl, 1-naphthyl, 2-naphthyl, and the like.

The term “cycloalkenyl”, alone or in combination, means a cycloalkyl substituent having one or more double bonds. Examples of cycloalkenyl substituents include, but are not limited to, cyclopentenyl, cyclohexenyl, cyclooctenyl, cyclopentadienyl, cyclohexadienyl and cyclooctadienyl.

The terms “cyclic”, “cycle” or “cycylyl” means a ring structure containing 3 to 20 carbon atoms, preferably 5 to 10 carbon atoms, which may be heterocyclic. The cyclic, cycle or cycylyl can also contain more than one ring

The term “cycloalkenylalkyl” means an alkyl substituent as defined above which is substituted by a cycloalkenyl substituent as defined above. Examples of cycloalkenylalkyl substituents include, but are not limited to, 2-cyclohexen-1-ylmethyl, 1-cyclopenten-1-ylmethyl, 2-(1-cyclohexen-1-yl)ethyl, 3-(1-cyclopenten-1-yl)propyl, 1-(1-cyclohexen-1-ylmethyl)pentyl, 1-(1-cyclopenten-1-yl)hexyl, 6-(1-cyclohexen-1-1-yl)hexyl, 1-(1-cyclopenten-1-yl)nonyl and 1-(1-cyclohexen-1-yl)nonyl.

The term “cycloalkyl”, alone or in combination means a cycloalkyl radica containing from 3 to about 10, preferably from 3 to about 8, and most preferably from 3 to about 6, carbon atoms. Examples of such cycloalkyl substituents include, but are not limited to, cyclopropyl, cyclobutyl, cyclopentyl, cyclohexyl, cycloheptyl, cyclooctyl, and perhydronaphthyl.

The term “cycloalkylalkyl” means an alkyl substituent as defined above which is substituted by a cycloalkyl substituent as defined above. Examples of cycloalkylalkyl substituents include, but are not limited to, cyclohexylmrthyl, cyclopentylmethyl, (4-isopropylcyclohexyl)methyl, (4-t-butyl-cyclohexyl)methyl, 3-cyclohexylpropyl, 2-cyclohexylmethylpentyl, 3-cyclopentylmethylhexyl, 1-(4-neopentylcyclohexyl)methylhexyl, and 1-(4-isopropylcyclohexyl)methylheptyl.

The term “cycloalkylcycloalkyl” means a cycloalkyl substituent as defined above which is substituted by another cycloalkyl substituent as defined above. Examples of cycloalkylcycloalkyl substituents include, but are not limited to, cyclohexylcyclopentyl and cyclohexylcyclohexyl.

The term “halide” means chloride, fluoride, iodide, or bromide.

The term “heterocyclic”, “heterocycle” or “heterocycylyl” means a cyclic, cycle or cycylyl containing at least one other kind of atom, in addition to carbon, in the ring. Such atoms include, but are not limited to, nitrogen, oxygen and sulfur. The heterocyclic can also contain more than one ring. Examples of heterocyclics include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups.

The term “R groups” means the group of variable substituents designated as “R” attached to the carbon atoms of the macrocycle, i.e., R1, R′1, R2, R′2, R3, R′3, R4, R′4, R5, R′5, R6, R′6, R7, R′7, R8, R′8, R9, R′9, R10, and R′10.

The term “saturated, partially saturated or unsaturated cycle or heterocycle” means a fused ring structure in which 2 carbons of the ring are also part of the fifteen-membered macrocyclic ligand in which the ring can contain no double bonds (in the case of a saturated ring structure) or at least one double bond, which may be conjugated or unconjugated with another double bond. The ring structure can contain 3 to 20 carbon atoms, preferably 5 to 10 carbon atoms, which may be heterocyclic. The cyclic can also contain more than one ring.

The term “organic acid anion” refers to carboxylic acid anions having from about 1 to about 18 carbon atoms.

The term “patient” or “subject” as used herein is intended to refer to an individual having a disease or condition or predisposed to having a disease or condition involving inflammation. In particular, the “patient” or “subject” can be a mammal although other vertebrates such as avian species can also be included with the meaning of the terms. It is envisioned that a mammal patient to which the catalyst for the dismutation of superoxide in combination with a corticosteroid wilt be administered, in the methods or compositions of the invention, will be a human. However, other mammal patients in veterinary (e.g., companion pets and large veterinary animals) and other conceivable contexts are also contemplated.

As used herein, the terms “treatment” or “treating” relate to any treatment of any inflammatory disease or disorder or condition and include: (1) preventing-inflammatory disease from occurring in a subject; (2) inhibiting the progression or initiation of the inflammatory disease, i.e., arresting or limiting its development; or (3) ameliorating or relieving the symptoms of the inflammatory disease.

The term “inflammatory disease” or “inflammatory disorder” or “inflammatory condition” refers to any disease or condition marked by inflammation, which can be caused by any one or more of a multitude of inciting events, including radiant, mechanical, chemical, infections, and immunological stimuli or which can be idiopathic or which may be treated with corticosteroids. Some inflammatory diseases, disorders and conditions include, but are not limited to, arthritis, inflammatory bowel disease, asthma, psoriasis, organ transplant rejections, radiation-induced injury, cancer, lupus and other autoimmune disorders, fibromyalgia, systemic lupus erythematosus, scleroderma, juvenile rheumatoid arthritis, ankylosing spondylitis, Sjogren's syndrome, gout, infectious arthritis, reactive arthritis, psoriatic arthritis, bursitis, tendonitis, burns, trauma, stroke, rheumatic disorders, renal diseases, allergic diseases, infectious diseases, ocular diseases, skin diseases, gastrointestinal diseases, hepatic diseases, cerebral edema, sarcoidosis, thrombocytopenia, spinal cord injury, autoimmune disorders, or any other disease, disorder or condition that may be treated with corticosteroids

The term “arthritis” refers to inflammation of the joints and refers to a group of more than 100 rheumatic diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis (“OA”) and rheumatoid Arthritis (“RA”). RA is the most severe of these two forms in terms of pain; while OA is the most common form. RA differs from OA in that RA involves an initial inflammation of the lining of the joint whereas OA is not initiated by joint inflammation although inflammation can develop during the course of the disease. Inflammatory diseases involving bone and/or joint include, in particular, rheumatoid arthritis, osteoarthritis (degenerative joint disease), fibromyalgia, systemic lupus erythematosus, scleroderma (systemic sclerosis), juvenile rheumatoid arthritis, ankylosing spondylitis, Sjogren's syndrome, gout, infectious arthritis, reactive arthritis (Reiter's syndrome), psoriatic arthritis, bursitis and tendonitis.

The term “precursor ligand” means the organic ligand of a SOD mimic without the chelated transition metal cation and charge neutralizing anions.

The term “therapeutically effective amounts” means those amounts that, when administered to a particular subject in view of the nature and severity of that subject's disease or condition, will have the desired therapeutic effect, e.g., an amount which will cure, or at least partially arrest or inhibit the disease or condition.

As used herein the term “substantially effective” with respect to treating or preventing the inflammatory disease need not necessarily mean a complete prevention or a complete cure of the inflammatory disease or symptoms thereof. Instead, the terms are intended to refer to a meaningful amelioration of disease and/or symptoms of the disease. For example, in Example 2 below it is shown that the combination of a non-proteinaceous catalyst for the dismutation of oxygen and dexamethasone significantly reduced histological damage score from a value of about 2.5 to 3.0 to a value of about 1 although a score of 0 would have been assigned to no bone damage. The damage scores were assigned as follows: 1=tissue swelling and edema, 2=joint erosion, and 3=bone erosion and osteophyte formation. Thus, it would be expected that the combination treatment would substantially prevent joint erosion, bone erosion and osteophyte formation, whereas some tissue swelling and edema might still be seen in at least some of the subjects. Conversely, the term “not substantially effective” or “substantially ineffective” is intended to mean that no meaningful prevention or amelioration of inflammation or symptoms thereof is produced.

The term “joint” or “joints” refers to the place of union or junction between two or more bones of the skeleton.

The term “co-administration” means the administration of at least two agents to a subject on the same or different treatment regimes, at the same time, i.e. simultaneously, on overlapping treatment schedules or sequentially. Such administration can, in certain instances, provide beneficial effects of the combination of two or more agents.

The combination of a non-proteinaceous catalyst for the dismutation of superoxide and a corticosteroid can be administered in two preparations, i.e. in separate formulations, such as, for example in sequence or simultaneously or in overlapping treatment regimens, or in one preparation, i.e. a single formulation for the prevention or treatment of an inflammatory disease. The compositions of this invention can be administered to the subject subcutaneously, intravenously, or intramuscularly. In a preferred embodiment; the compositions of this invention are administered to a subject subcutaneously or intramuscularly.

Some corticosteroids useful for this invention include, but are not limited to, cortisol, cortisone, hydrocortisone fludrocortisone, prednisone, prednisolone, 6-methylprednisolone, triamcinolone, betamethasone, and dexamethasone. However, any of the adrenal corticosteroid hormones isolated from the adrenal cortex or produced synthetically, and derivatives thereof that are used for treatment of inflammation are useful for this invention. The table below lists several corticosteroids and provides their relative potencies and equivalent doses.

TABLE 1
Relative Potencies and Equivalent Doses
of Representative Corticosteroids

| | Anti- | Na⁺- | | |
|---|---|---|---|---|
| | inflammatory | retaining | Duration | Equivalent |
| Compound | Potency | Potency | (T_{1/21} Hours) | Dose (mg) |
| Cortisol | 1 | 1 | 8-12 | 20 |
| Cortisone | 0.8 | 0.8 | 8-12 | 25 |
| Fludrocortisone | 10 | 12.5 | 8-12 | N/A |
| Prednisone | 4 | 0.8 | 12-36 | 5 |
| Prednisolone | 4 | 0.8 | 12-36 | 5 |
| 6a- | 5 | 0.5 | 12-36 | 4 |
| Methyl- | | | | |
| prednisolone | | | | |
| Triamcinolone | 5 | 0 | 12-36 | 4 |
| Betamethasone | 25 | 0 | 36-72 | 0.75 |
| Dexamethasone | 25 | 0 | 36-72 | 0.75 |

The beneficial effect on inflammation of the combination of a non-proteinaceous catalyst for the dismutation of superoxide and a corticosteroid is shown in the examples section below. While not intending to be bound by any particular theory for the beneficial effect of the combination, one particular advantage of this invention is that the use of SOD mimics in combination with corticosteroids enhances the efficiency of the corticosteroids in the treatment of inflammatory diseases and thereby allowing the use of a lower dosage of corticosteroids and decreasing the risk of side effects associated with corticosteroids. Glucocorticoids and their receptors become deactivated when exposed to superoxide and other free radicals, thereby forcing an increase in the dosage of glucocorticoids to have the desired therapeutic effect. In fact, administration of antioxidants to LPS treated RAW cells prevents the inactivation of dexamethasone as shown in Example 3. The dosage of corticosteroid needed for treatment of inflammatory disease is decreased by at least about 1%, more preferably by at least 10%, even more preferably by at least 25%, and most preferably by at least 50% when used in combination with the catalysts for dismutation of superoxide of this invention.

It is also known that SOD mimics produce anti-inflammatory effects to attenuate the degree of chronic inflammation, tissue damage, and bone damage associated with collagen induced arthritis in the rat (Salvemini et al., Arthritis & Rheumatism 44:2909-2921, 2001). Thus, these agents can be therapeutically effective in the management of chronic inflammatory diseases such as rheumatoid arthritis. Hence, another aspect of the beneficial effect of the combination can be the anti-inflammatory actions of the SOD mimics and corticosteroids taken together. Thus, the synergism associated with the combined use of SOD mimics and corticosteroids provides substantial advantages for the treatment of inflammatory diseases.

Preferably, the compound employed in the method of the present invention will comprise a non-proteinaceous catalyst for the dismutation of superoxide anions (“SOD mimic”) as opposed to a native form of the SOD enzyme. As utilized herein, the term “SOD mimic” means a low-molecular-weight catalyst for the conversion of superoxide anions into hydrogen peroxide and molecular oxygen. These catalysts consist of an organic ligand having a pentaazacyclopentadecane portion and a chelated transition metal ion, preferably manganese or iron. The term may include catalysts containing short-chain polypeptides (under 15 amino acids), or macrocyclic structures derived from amino acids, as the organic ligand. The term explicitly excludes a SOD enzyme obtained from any natural sources. SOD mimics are useful in the method of the present invention as compared to native SOD because of the limitations associated with native SOD therapies such as, solution instability, limited cellular accessibility due to their size, immunogenicity, bell-shaped dose response curves, short half-lives, costs of production, and proteolytic digestion. See, e.g., Salvemini et al., Science 286: 304-306 (1999). For example, the best known native SOD, CuZn, has a molecular weight of 33,000 kD. In Contrast, SOD mimics have an approximate molecular weight of 400 to 600 Daltons.

In a particularly preferred embodiment, the SOD mimics utilized in the present invention comprise an organic ligand chelated to a metal ion. Particularly preferred catalysts are pentaaza-macrocyclic ligand compounds, more specifically the copper, manganese(II), manganese (III), iron(II) and iron(III) chelates of pentaazacyclopentadecane compounds. The pentaaza macrocyclic ligand complexes of Mn(II) are particularly advantageous for use in the present invention because, in addition to having a low molecular weight, they are highly selective for the dismutation of superoxide anions and possess catalytic rates similar to or faster than native SOD counterparts. Examples of this class of SOD mimic, M40403 and M40401, are set forth in the examples below. These pentaazacyclopentadecane compounds can be represented by the following formula: 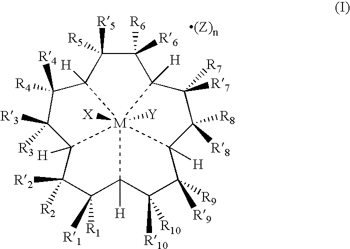
wherein

(i) one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ are independently:

(ia) hydrogen; or

(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino acids, wherein R11 and R12 are independently hydrogen or alkyl; and

(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and

(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰, together with the carbon atom to which they are attached independently form a substituted or unsubstituted and saturated, partially saturated, or unsaturated cycle or heterocycle having 3 to 20 carbon atoms; and

(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(v) optionally, one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, which is attached to a different carbon atom in the macrocyclic ligand may be bound to form a strap represented by the formula:
—(CH₂)_{I}—Q—(CH₂)_{J}—R—(CH₂)_{K}—S—(CH₂)_{L}—
wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vi) combinations of any of (i) through (v) above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge neutralizing anions which are derived from any monodentate or polydentate coordinating ligand and a ligand system and the corresponding anion thereof; or

X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰; and

n is an integer from 0 to 3.

Particular ligands from which X, Y and Z can be selected include halide, organic acid, nitrate and bicarbonate anions.

The “R” groups attached to the carbon atoms of the macrocycle can be in the axial or equatorial position relative to the macrocycle. When the “R” group is other than hydrogen or when two adjacent “R” groups, i.e., on adjacent carbon atoms, together with the carbon atoms to which they are attached form a saturated, partially saturated or unsaturated cyclic or a nitrogen containing heterocycle, or when two R groups on the same carbon atom together with the carbon atom to which they are attached form a saturated, partially saturated or unsaturated ring structure, it is preferred that at least some of the “R” groups are in the equatorial position for reasons of improved activity and stability. This is particularly true when the complex contains more than one “R” group which is not hydrogen.

One of the particular compounds of this class of pentaaza-macrocyclic class is designated M40401 and is represented by the following formula:

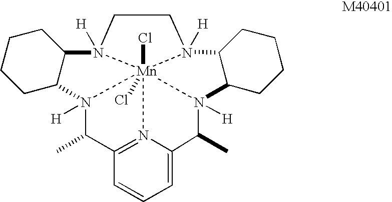

Another particular compound of this class of pentaaza-macrocyclic class is designated M40403 and is represented by the following formula:

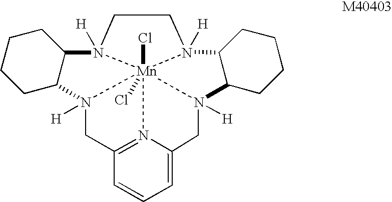

A wide variety of pentaaza-macrocyclic ligand compounds with superoxide dismutating activity may be synthesized. The transition metal center of the catalyst is thought to be the active site of catalysis, wherein the manganese or iron ion cycles between the (II) and (III) states.

The pentaaza-macrocyclic ligand compound catalysts described have been further described in U.S. Pat. Nos. 5,637,578, 6,214,817, and PCT application W098/58636, all of which are hereby incorporated by reference. These pentaaza-macrocyclic ligand catalysts may be produced by the methods disclosed in U.S. Pat. No. 5,610,293.

Iron or manganese porphyrins are also suitable non-proteinaceous catalysts for use in the present invention, such as, for example, MnIII tetrakis(4-N-methylpyridyl)porphyrin, MnIII tetrakis-o-(4-N-methylisonicotinamidophenyl) porphyrin, MnIII tetrakis(4-N-N-N-trimethylanilinium)porphyrin, MnIII tetrakis(1-methyl-4-pyridyl)porphyrin, MnIII tetrakis(4-benzoic acid porphyrin, MnII octabromo-meso-tetrakis(N-methylpyridinium-4-yl)porphyrin, 5,10,15,20-tetrakis (2,4,6-trimethyl-3,5-disulfonatophenyl)-porphyrinato iron (III) (FeTMPS), Felll tetrakis(4-N-methylpyridyl)porphyrin, and Felll tetrakis-o-(4-N-methylisonicotinamidophenyl)porphyrin and preferably, substituted iron porphyin 5,10,15,20-tetrakis (2,4,6-trimethyl-3,5-disulfonatophenyl)-porphyrinato iron (III) (FeTMPS) may also be used in the methods and compositions of the present invention which can be seen in U.S. Pat. No. 6,103,714, herein incorporated by reference in its entirety. The catalytic activities and methods of purifying or synthesizing these non-proteinaceous catalysts are well known in the organic chemistry arts.

Activity of the porphyrin compounds or complexes of the present invention for catalyzing the dismutation of superoxide can be demonstrated using the stopped-flow kinetic analysis technique as described in Riley, D. P. et al., Anal. Biochem., 196: 344-349 (1991) which is incorporated herein by reference. The stopped-flow kinetic analysis is suitable for screening compounds for SOD activity and activity of the porphyrin compounds or complexes of the present invention, as shown by stopped-flow analysis, correlate to treating the above disease states and disorders. However, the stopped-flow analysis is not an appropriate method for demonstrating the activity of all superoxide dismutase mimics. Other methods may be appropriate or preferred for some SOD mimics. See Weiss et al, Evaluation of Activity of Putative Superoxide Dismutase Mimics. Direct Analysis by Stopped-flow Kinetics, J. Biol. Chem. 268(31): 23049-54 (Nov. 5, 1993).

Contemplated equivalents of the general formulas set forth above for the compounds and derivatives as well as the intermediates are compounds otherwise corresponding thereto and having the same general properties such as tautomers of the compounds and such as wherein one or more of the various R groups are simple variations of the substituents as defined therein, e.g., wherein R is a higher alkyl group than that indicated, or where the tosyl groups are other nitrogen or oxygen protecting groups or wherein the O-tosyl is a halide. Anions having a charge other than 1, e.g., carbonate, phosphate, and hydrogen phosphate, can be used instead of anions having a charge of 1, so long as they do not adversely affect the overall activity of the complex. However, using anions having a charge other than 1 will result in a slight modification of the general formula for the complex set forth above. In addition, where a substituent is designated as, or can be, a hydrogen, the exact chemical nature of a substituent which is other than hydrogen at that position, e.g., a hydrocarbyl radical or a halogen, hydroxy, amino and the like functional group, is not critical so long as it does not adversely affect the overall activity and/or synthesis procedure. Further, it is contemplated that manganese(III) complexes will be equivalent to the subject manganese(II) complexes.

For use in treatment or prophylaxis of subjects, the compounds of the invention can be formulated as pharmaceutical or veterinary compositions. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage of a catalyst for the dismutation of superoxide in combination with at least one corticosteroid. The catalyst for the dismutation of superoxide is preferably a SOD mimetic, as described in more detail above. The SODm's of this invention, as well as the corticosteroids of this invention, are preferably used in combination with a pharmaceutically acceptable carrier, either in the same formulation or in separate formulations.

The doses of the corticosteroid and the non-proteinaceous catalyst for the dismutation of superoxide will depend upon a number of factors including the subject being treated, the particular agent administered, the route of administration and the like all. For example, based on the studies in rats reported with the combination of dexamethasone and M40303, the corticosteroid can be administered parenterally to a human subject at doses of from about 0.000005 to about 0.5 mg/kg, from about 0.000015 to about 0.15 mg/kg, from about 0.00015 to about 0.015 mg/kg, from about 0.0005 to about 0.005 mg/kg or about 0.0015 mg/kg and the catalyst can administered parenterally at a dose of from about 0.0025 to about 25 mg/kg, from about 0.025 to about 2.5 mg/kg, from about 0.075 to about 0.75 mg/kg or about 0.25 mg/kg. In particular, the combination of dexamethasone and M40303 can be administered parenterally to human subjects at a dose of dexamethasone of about 0.0015 mg/kg and a dose of M40303 of about 0.25 mg/kg.

The compositions of the present invention may be incorporated in conventional pharmaceutical formulations (e.g., injectable solutions) for use in treating humans or animals in need thereof. Pharmaceutical compositions can be administered by a variety of regimens, for example, subcutaneously, intravenously, or intramuscularly by rapid or bolus injection, or by infusion of large volume parenteral solutions or the like. The term parenteral as used herein includes subcutaneous, intravenous, intramuscular or intrasternal administration by injection, or infusion techniques.

For example, a parenteral therapeutic composition may comprise a sterile isotonic saline solution containing between 0.1 percent and 90 percent weight to volume of the catalysts for the dismutation of superoxide. A preferred solution contains from about 5 percent to about 25 weight percent catalysts for dismutation of superoxide in solution (% weight per volume). The parenteral therapeutic composition may contain, in addition to the isotonic saline solution and a catalyst for the dismutation of superoxide, at least one corticosteroid at between 1:100 to 100:1 weight ratio of the corticosteroid to the catalyst for the dismutation of superoxide. A preferred solution contains approximately 1:10 to 10:1 weight ratio of the corticosteroid to the catalyst for the dismutation of superoxide.

Alternatively, the corticosteroid may be administered sequentially to the catalyst for the dismutation of superoxide. The dosage of corticosteroid to be used may vary. A primary consideration for the dosage level of the corticosteroids of this invention is the monitoring of the known side effects in an individual.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The preparations may, if desired, be presented in a pack or dispenser device which may contain one or more unit dosage forms containing the catalyst for the dismutation of superoxide in conjunction with at least one corticosteroid. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers having therapeutically or prophylactically effective amounts of the catalyst and corticosteroid combination in pharmaceutically acceptable form. The catalyst and corticosteroid combination in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the combination, and/or a packaged alcohol pad. Instructions are optionally included for administration of the catalyst and corticosteroid combination by a clinician or by the patient.

The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The pharmaceutical compositions of the present invention are preferably administered to a human. However, besides being useful for human treatment, these compositions are also useful for veterinary treatment of companion animals, exotic animals and farm animals, including mammals, rodents, avians, and the like. More preferred of non-human animals include horses, dogs, cats, sheep, and pigs.

The detailed description set forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

Without further elaboration, it is believed that one skilled in the art can, using the preceding description, utilize the present invention to its fullest extent. The following examples are, therefore, to be construed as merely illustrative, and not limitative of the remainder of the disclosure in any way whatsoever.

### EXAMPLE 1

This example illustrates the effects of dexamethasone and M40403 in a Rodent Model of Collagen-Induced Arthritis

Objective. The objective of these studies were to determine whether low doses of M40403 potentiate the effects of low dose dexamethasone in the rat model of collagen induced arthritis.

Methods. Collagen-induced arthritis (CIA) was induced in Lewis rats by an intradermally injection of 100 μl of the emulsion (containing 100 μg of bovine type II collagen) (II) and incomplete Freund's adjuvant (IFA) at the base of the tail. On day 21, a second injection of CII in incomplete Freund's adjuvant was administered.

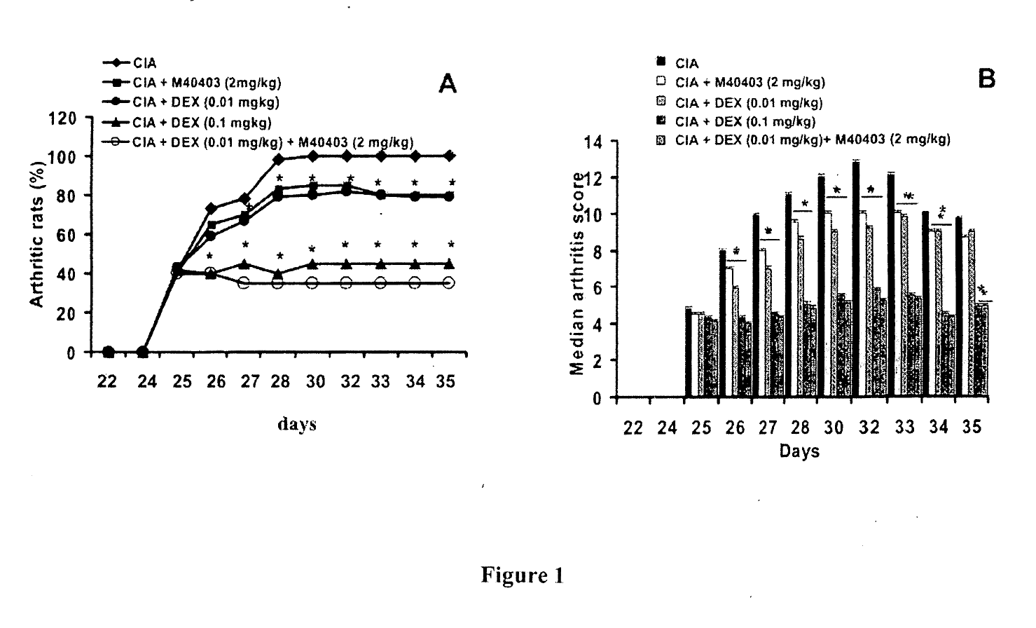

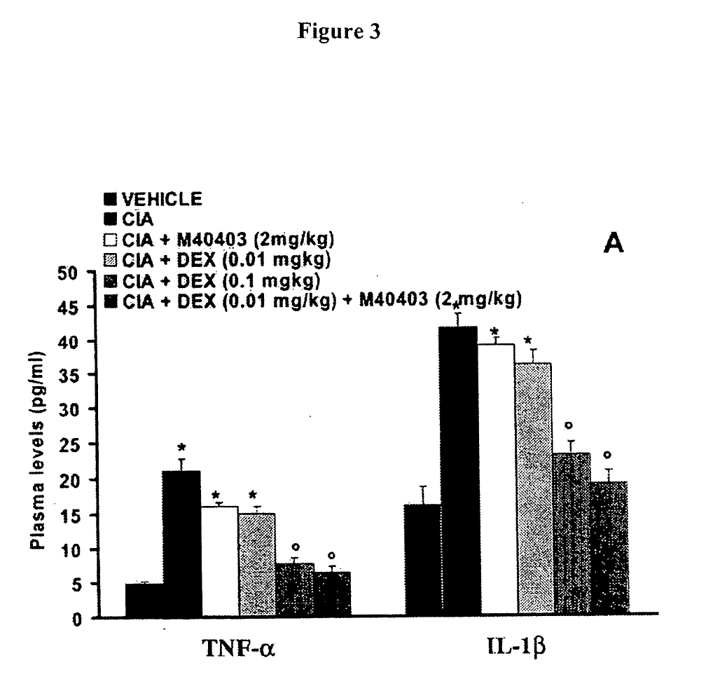

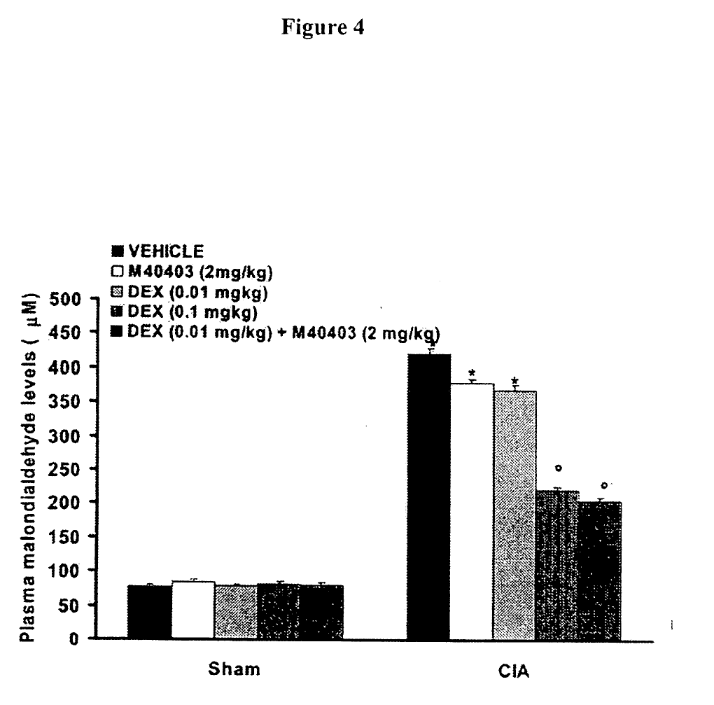

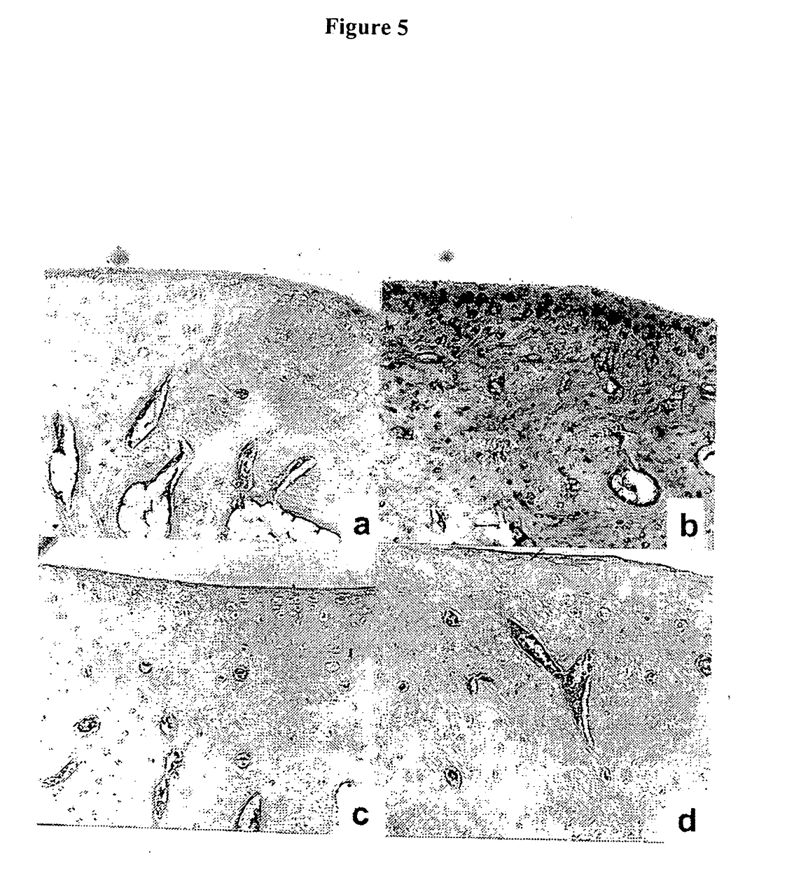

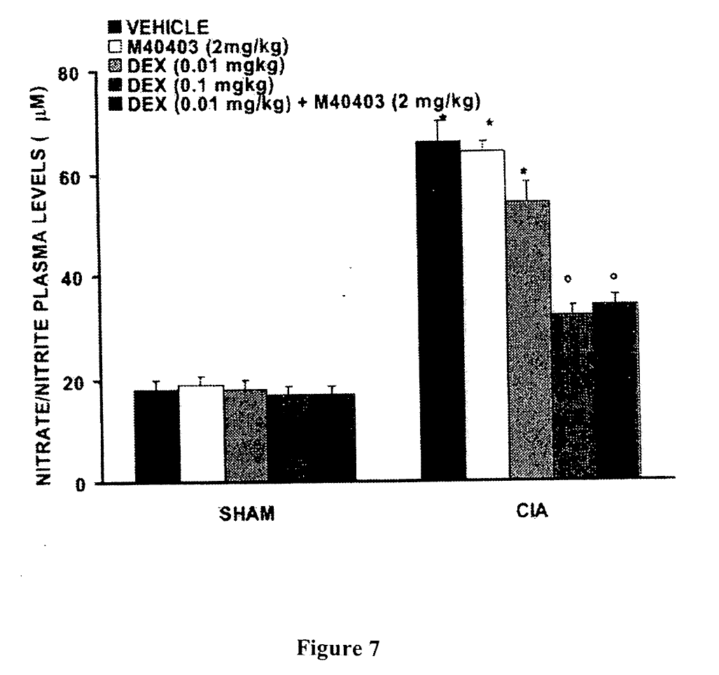

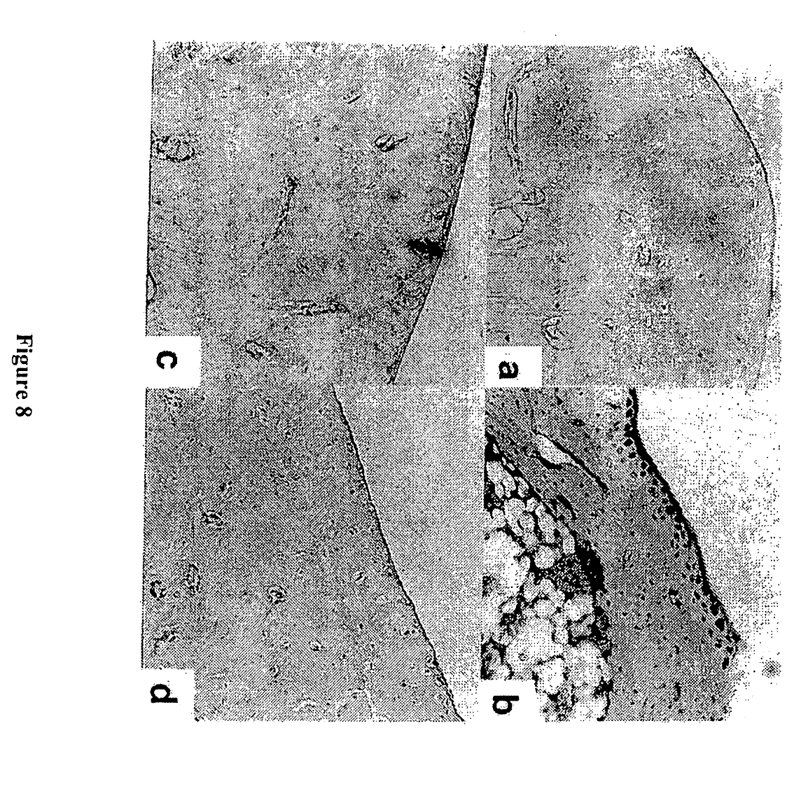

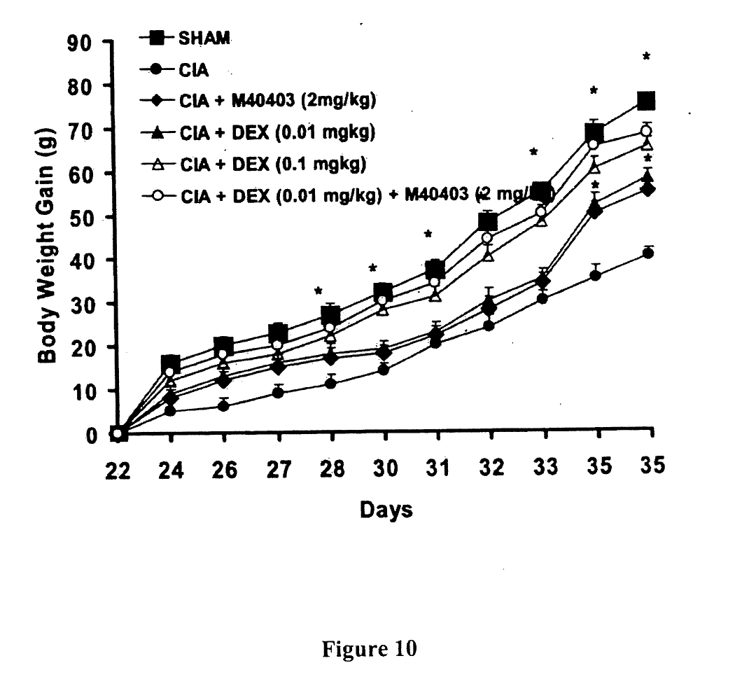

Results. Lewis rats developed an erosive hind paw arthritis when immunized with an emulsion of CII in IFA. Macroscopic clinical evidence of CIA first appeared as periarticular erythema and edema in the hind paws by day 24-26 after the first injection as shown in FIG. 1. The incidence of CIA was 100% by day 27 in the CII challenged rats; and CIA severity progressed over a 35-day period as shown in FIG. 1. A marked increase in the plasma levels of TNF-α and IL-1β as shown in FIG. 3, malonylaldehyde (MDA, a marker of lipid peroxidation) as shown in FIG. 4, and nitric oxide (NO) as shown in FIG. 7 was observed at day 35. Immunohistochemical analysis for nitrotyrosine (a marker for peroxynitrite formation) and PARS (a nuclear enzyme activated by DNA single strand damage) revealed a positive staining in inflamed joints from collagen-treated rats suggestive of the formulation of peroxynitrite and DNA damage as shown in FIG. 5. Immunohistochemical analysis for the inducible nitric oxide synthase and cyclooxygenase (iNOS and COX-2) revealed a positive staining in inflamed joints from collagen-treated rats as shown in FIG. 8. Treatment of rats with low does of M40403 (2 mg/kg daily, given by intraperitoneal injection) or a low dose of dexamethasone (0.01 mg/kg given daily orally) starting at the onset of arthritis (day 25), ameliorated the extent of the arthritic response (as defined by assessing the parameters described above) by some 10-20%. On the other hand, when these two low doses were combined the extent of the protective effects reached some 60-90%. The degree of protection observed with combination of these low doses was similar to the one attained with dexamethasone at 0.1 mg/kg. Finally, arthritic rats treated with combination of low doses of DEX (0.01 mg/kg) and M40403 (2 mg/kg) or with DEX at the high dose (0.1 mg/kg) gained weight at the same rate and to the same extent as normal non-arthritic rats as shown in FIG. 10.

Conclusion. The study provides the first evidence that M40403, enhances the anti-inflammatory effects of dexamethasone in collagen-induced arthritis in the rat.

Materials and Methods

Animals: Male Lewis rats (weighing approximately 160-180 g and purchased from Charles River; Milan; Italy) were housed in a controlled environment and provided with standard rodent chow and water.

Experimental Protocol: Animals were randomly divided into six groups (n=10 for each group) as follows (CIA refers to Collagen-Induced Arthritis; DEX refers to dexamethasone):
(1) Sham group: Rats received intraperitoneally (i.p.) a vehicle of 26 mM sodium bicarbonate buffer, pH 8.1-8.3;
(2) CIA alone: In this group rats were subjected to CIA without receiving treatment with M40303 or DEX;
(3) CIA+M40403: In this group rats were subjected to CIA were treated with M40403 at 2 mg/kg i.p. every 24 hours, starting from day 25;
(4) CIA-DEX 0.01: In this group rats subjected to CIA were treated orally with DEX at 0.01 mg/kg starting from day 2;
(5) CIA+DEX 0.1: In this group rats subjected CIA were treated orally with DEX at 0.1 mg/kg starting from day 25.
(6) CIA+DEX+M40403: In this group rats subjected to CIA were treated with DEX (0.01 mg/kg, orally) and with M40403 (2 mg/kg, i.p.) starting from day 25.

Induction of Collagen-Induced Arthritis: Bovine type II collagen (CII) was dissolved in 0.01 M acetic acid at a concentration of 2 mg/ml by stirring overnight at 4° C. Dissolved CII was frozen at −70° C. until use. Incomplete Freund's adjuvant (IFA) was prepared by the addition of Mycobacterium tuberculosis H37Ra at a concentration of 2 mg/ml. Before injection, CII was emulsified with an equal volume of IFA. Collagen-induced arthritis was induced as previously described. On day 1, Lewis rats were injected intradermally at the base of the tail with 100 μl of the emulsion (containing 100 μg of CII). On day 21, a second injection of CII in IFA was administered.

Clinical Assessment of CIA: Rats were evaluated daily for arthritis by using a macroscopic scoring system: 0=no signs of arthritis; 1=swelling and/or redness of the paw or one digit; 2=two joints involved; 3=more than two joints involved; and 4=severe arthritis of the entire paw and digits. Arthritic index for each rat was calculated by adding the four scores of individual paws. Clinical severity was also determined by quantitating the change in the paw volume using plethysmometry (model 7140; Ugo Basile).

Immunohistochemical localization of Nitrotyrosine, PARS, COX-2 and iNOS: At day 35, the joints organs were then trimmed, placed in decalcifying solution for 24 hours and 8 μm sections were prepared from paraffin embedded tissues. After deparaffinization, endogenous peroxidase was quenched with 0.3% H₂O₂ in 60% methanol for 30 minutes. The sections were permeabilized with 0.1% Triton X-100 in PBS for 20 minutes. Non-specific adsorption was minimized by incubating the section in 2% normal goat serum in phosphate buffered saline for 20 minutes. Endogenous biotin or avidin binding sites were blocked by sequential incubation for 15 minutes with avidin and biotin. Sections were incubated overnight with 1) anti-rabbit polyclonal antibody directed at iNOS (1:1000 in PBS, v/v) (DBA, Milan, Italy) or 2) with anti-COX-2 goat polyclonal antibody (1:500 in PBS, v/v) or 3) with anti-nitrotyrosine rabbit polyclonal antibody (1:1000 in PBS, v/v) or 4) with anti-poly (ADP-Ribose) goat polyclonal antibody rat (1:500 in PBS, v/v). Controls included buffer alone or non-specific purified rabbit IgG. Specific labeling was detected with a biotin-conjugated goat anti-rabbit IgG (for nitrotyrosine and iNOS) or with a biotin-conjugated goat anti-rabbit IgG (for PARS and COX-2) and avidin-biotin peroxidase complex. In order to confirm that the immunoreaction for the nitrotyrosine was specific some sections were also incubated with the primary antibody (anti-nitrotyrosine) in the presence of excess nitrotyrosine (10 mM) to verify the binding specificity. To verify the binding specificity for PARS, COX-2 and iNOS, some sections were also incubated with only the primary antibody (no secondary) or with only the secondary antibody (no primary). In these situations, no positive staining was found in the sections indicating that the immunoreaction was positive in all the experiments carried out.

Measurement of nitrite/nitrate: Plasma levels of nitrite/nitrate were measured as an indicator of NO synthesis. Briefly, the nitrate in the supernatant was first reduced to nitrite by incubation with nitrate reductase (670 mU/ml) and NADPH (160 μm) at room temperature for 3 hours. The nitrite concentration in the samples was then measured by the Griess reaction, by adding 100 μl of Griess reagent (0.1 naphthylethylendiamide dihydrochloride in H₂O and 1% sulphanilamide in 5% concentrated H₃PO₄; vol. 1:1) to 100 μl samples. The optical density at 55 nm (OD₅₅₀) was measured using ELISA microplate reader (SLT-Labinstruments Salzburg, Austria). Nitrate concentrations were calculated by comparison with OD₅₅₀ of standard saline solutions.

Malondialdehyde (MDA) Measurement: Plasma levels of malondialdehyde (MDA), i.e. thiobarbituric acid-reactant substances, were determined as an indicator of lipid peroxidation. An aliquot (100 μl) of the plasma collected at the specified time was added to a reaction mixture containing 200 μl of 8.1% SDS, 1500 μl of 20% acetic acid (pH 3.5), 1500 μl of 0.8% thiobarbituric acid and 700 μl distilled water. Samples were then boiled for 1 hour at 95° C. and centrifuged at 3,000 g for 10 minutes. The OD₆₅₀ was measured using an ELISA microplate reader. Levels of MDA were calculated by comparison with the OD₆₅₀ of standard solutions of 1,1,3,3-tetramethoxypropane (malonaldehyde bis[dimethyl acetal]). The absorbance of the supernatant was measured by spectrophotometry at 650 nm.

Measurement of Cytokines: TNF-α and IL-1β levels were evaluated in plasma at 35 days after the induction of arthritis. The assay was carried out by using a colorimetric, commercial kit (Calbaiochem-Novabiochem Corporation, USA). The ELISA has a lower detection limited of 5 pg/ml.

Materials: Unless otherwise stated, all compounds were obtained from Sigma-Aldrich Company Ltd. (Poole, Dorset, UK). Thiopentone sodium (Intraval Sodium®) was obtained from Rhône Mérieux Ltd. (Harlow, Essex, UK). Biotin blocking kit, biotin-conjugated goat anti-rabbit IgG, Primary anti-nitrotyrosine, anti-poly (ADP-Ribose) synthetase antibodies primary anti-iNOS, anti-COX-2 and avidin-biotin peroxidase complex were obtained from DBA (Milan, Italy). All other chemicals were of the highest commercial grade available. All stock solutions were prepared in nonpyrogenic saline (0.9% NaCI; Baxter Healthcare Ltd., Thetford, Northfold, UK).

Data Analysis: All values in the figures and text are expressed as mean±standard error of the mean (SEM) of n observations. For the in vivo studies, n represents the number of animals studied. In the experiments involving histology or immunohistochemistry, the figures shown are representative of at least three experiments performed on different experimental days. Data sets were examined by one- and two-way analysis of variance, and individual group means were then compared with Student's unpaired t test. For the arthritis studies, Mann-Whitney U test (two-tailed, independent) was used to compare medians of the arthritic indices as reported earlier (Salvemini et al., Arthritis. Rheum. 44:2902-21, 2001). Values for the in vitro studies are presented as incidences (%), or medians. A p-value less than 0.05 was considered significant.

Results

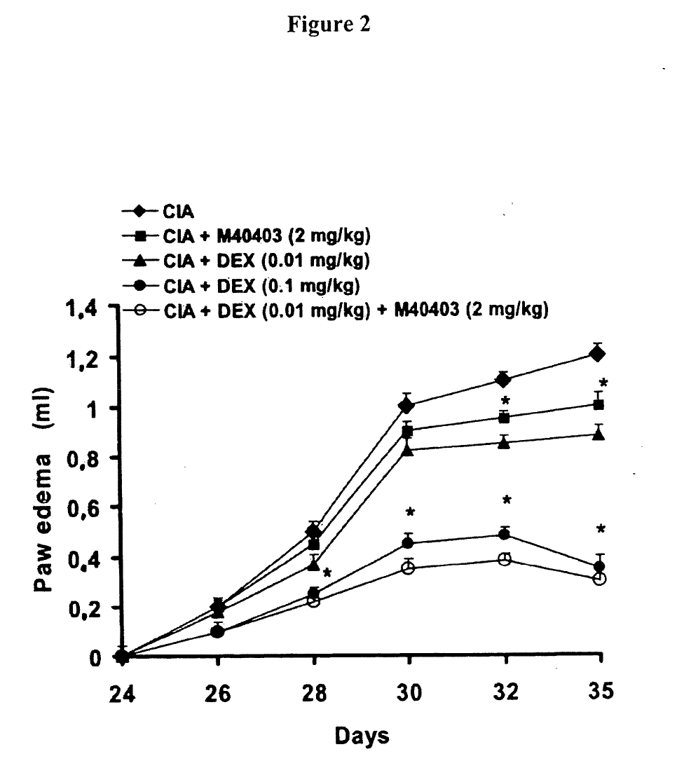

Effects of Combination therapy in the Development of Collagen-Induced Arthritis: CIA developed rapidly in rats immunized with CII and clinical signs (periarticular erythema and oedema) of the disease (FIG. 1A) first appeared in the hind paws between 24 and 26 days post-challenge. Furthermore, a 100% incidence of CIA was observed by day 28 in CII-immunized rats. Hind paw erythema and swelling increased in frequency and severity in a time-dependent mode with maximum arthritis indices of approximately 13 observed between 28 to 35 days post-immunization (FIG. 1B). When given at the low doses M40403 (2 mg/kg, i.p.) or DEX (0.01 mg/kg, p.o.) attenuated the development of CIA and arthritic score by some 10-20%. The maximum incidence of CIA in rats which received the high dose of DEX (0.1 mg/kg) was 45% (FIG. 1A, p<0.01). At this high dose, DEX (0.1 mg/kg) also exerted a significant suppression (p<0.01) of the arthritis index between days 26 and 35 post-CII immunization (FIG. 1B). In other words, the efficacy of a low dose of DEX at 0.01 mg/kg when given together with low dose of M40403 (2 mg/kg) was comparable to the efficacy of DEX at 0.1 mg/kg. Similar results were observed when assessing paw swelling (FIG. 2).

Effect of combination therapy of cytokine production and lipid peroxidation: At day 35, the levels of TNF-α and IL-1β were significantly elevated in the plasma from CIA-treated rats (FIG. 3). The degree of inhibition of TNF-α and IL-1β observed with a combination of low doses of DEX and M40403 (0.01 mg/kg and 2 mg/kg, respectively) was similar to that observed with DEX alone at the high dose (0.1 mg/kg) (FIG. 3). Similar results were observed when assessing plasma levels of MDA as an indicator of lipid peroxidation (FIG. 4).

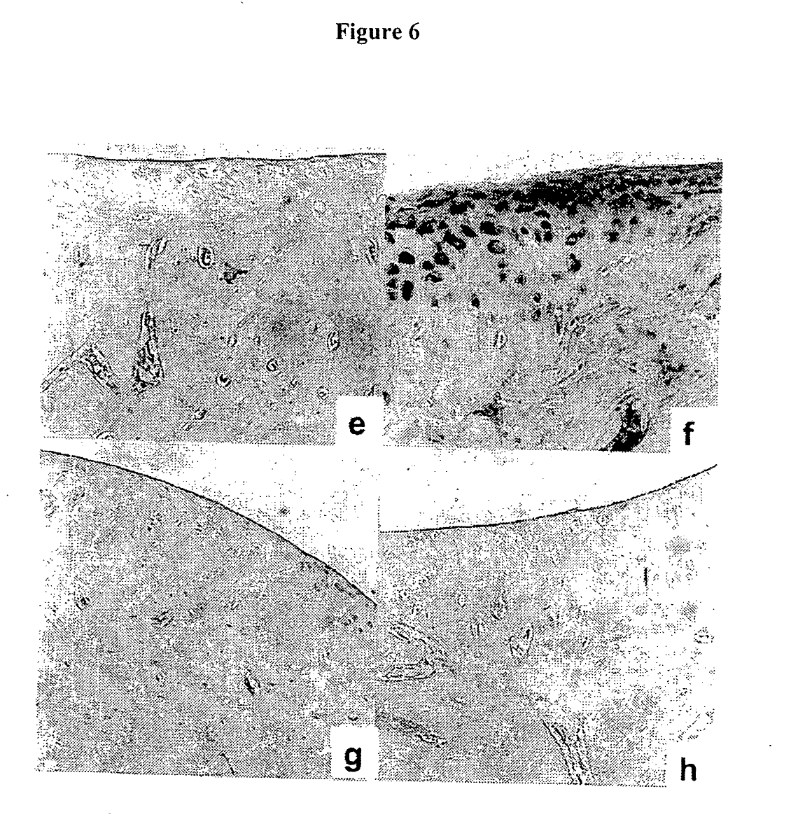

Nitrotyrosine formation and PARS activation: Immunohistochemical analysis and joint sections obtained from rats treated with collagen type II revealed a positive staining from nitrotyrosine and PARS, which was primarily localized in inflammatory cells (FIGS. 5B and 6B). No significant protective effect was observed in the group of animals treated with DEX (0.01 mg/kg) or with M40403 (2 mg/kg). In contrast, no positive nitrotyrosine or PARS staining was found in the joint of CIA-treated rats, which had been treated with the high dose of DEX alone (0.1 mg/kg; FIGS. 5C and 6C) or the combination therapy of low dose DEX and M40403, respectively (0.01 mg/kg+M40403 2 mg/kg; FIGS. 5D and 6D). There was no staining for either nitrotyrosine or PARS in joint obtained from sham-operated rats (FIGS. 5A and 6A).

Effect of combination therapy on NO production: At day 35 the levels of NOₓ were significantly elevated in the plasma from CIA-treated rats (FIG. 7). DEX at the highest dose (0.1 mg/kg) or the combination of low doses of DEX and M40403 (0.01 mg/kg and 2 mg/kg respectively) (FIG. 7) inhibited NOₓ to the same extent.

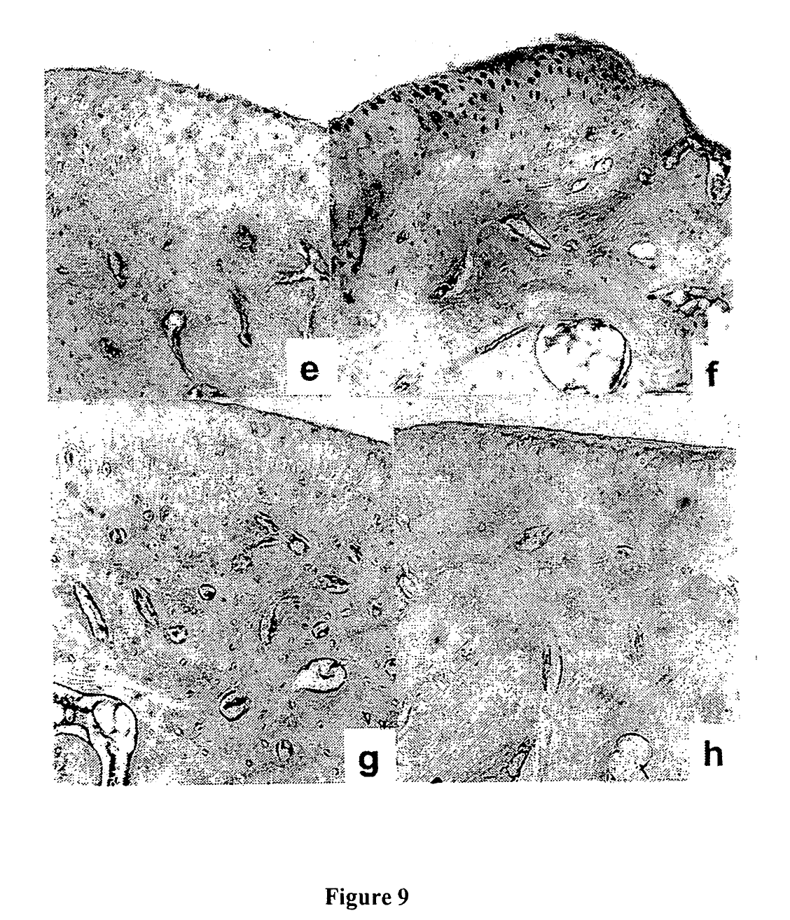

iNOS and COX-2 expression: Immunohistochemical analysis of joint sections obtained from rats treated with collagen type II revealed a positive staining for iNOS, and COX-2 which was primarily localized in inflammatory cells (FIGS. 8B and 9B). In contrast, no positive iNOS or COX-2 staining was found in the joints of CIA-treated rats, which had been treated with high dose of DEX (0.1 mg/kg; FIGS. 8C and 9C) or the combination of low dose DEX and M40403 (0.01 mg/kg and 2 mg/kg respectively; FIGS. 8D and 9D). No staining for iNOS or COX-2 was observed in joint obtained from sham-operated rats (FIGS. 8A and 9A). DEX (0.01 mg/kg) or M40403 (2 mg/kg) by themselves had no effect on iNOS or COX-2 staining.

Effects on body weight gain: The rate and the absolute gain in body weight were comparable in sham Lewis rats and CII-immunized rats for the first week (FIG. 10). Beginning on day 25, the collagen-challenged rats gained significantly less weight than the normal rats, and this trend continued through day 35. Rats treated with the high dose DEX (0.1 mg/kg) or the combination of the low dose DEX and M40403 (0.01 mg/kg and 2 mg/kg M40403 respectfully) gained weight at a rate that was similar to the one observed with sham animals (FIG. 10). Rats treated with low doses DEX (0.01 mg/kg) or M40403 (2 mg/kg) gained weight in a manner that was similar to CIA rats (FIG. 10).

### EXAMPLE 2

This example illustrates the effects of the combination of dexamethasone and M40403 on histology, structural morphometry and radiography of bone and joint tissues in the study described in Example 1.

Materials and Methods

Histologic examination. On day 35, animals were anesthetized and then killed and paws and knees were removed and fixed for histologic examination. Biopsy samples were fixed for 1 week in buffered formaldehyde solution (10% in phosphate buffered saline [PBS]) at room temperature, dehydrated by graded ethanol, and embedded in Paraplast (Sherwood Medical, Mahwah, N.J.). The paws were trimmed, placed in decalcifying solution for 24 hours, embedded in paraffin, and sectioned at 5 μm. Tissue sections were deparaffinized with xylene, stained with trichromatic van Gieson's stain, and studied using light microscopy (Dialux 22; Leitz, Wetzlar, Germany). In order to have a quantitative estimation of the damage to all paws and knees, sections (n=6 for each animal) were scored by 2 independent observers who were blinded to the experimental protocol. Morphologic changes were scored as follows: 0=no damage, 1=edema, 2=inflamed cell presence, 3 bone resorption.

Structural morphometry. Blinded histomorphometric analysis of the proximal tibia near the joint was performed on 5-elm-thick sections, using morphometry software, a computer with a digitizing board, and a Labophot microscope (Nikon, Melville, N.Y.) equipped with both visible and ultraviolet light sources and a camera lucida attachment (Zeiss, Milan, Italy). Parameters for histomorphometry used in this study, derived from Parfitt and colleagues (Parfitt et al., Bone histomorphometry: standardization of nomenclature symbols and units. J. Bone Miner Res. 2:595-610, 1987), and have been approved by an American Society for Bone and Mineral Research committee. To measure bone formation, osteoblast surface (ObS) was quantified relative to bone surface (Bs) (ObS/Bs). To measure bone resorption, eroded surface (ES) and osteoclast surface (OcS) were quantified relative to bone surface (ES/Bs and OcS/Bs, respectively).

Radiography. For radiography, rats were placed on a box 90 cm from the x-ray source, and normal and arthritic hind paws were radiographed with an X12 x-ray machine (Philips, Munich, Germany) using a 40-kW exposure for 0/01 second. An investigator who was blinded to the treatment regimen performed the radiographic scoring. The following radiographic scoring system was used: 0=no bone damage, 1=tissue swelling and edema, 2=joint erosion, and 3=bone erosion and osteophyte formation. This scoring system reflects the course of CIA in the rats which corresponds with the clinical onset of arthritis in humans. Thus, tissue swelling and edema occurs and within a few days of onset, erosion of cartilage and subchondral bone by pannus-like tissue is evident.

Results

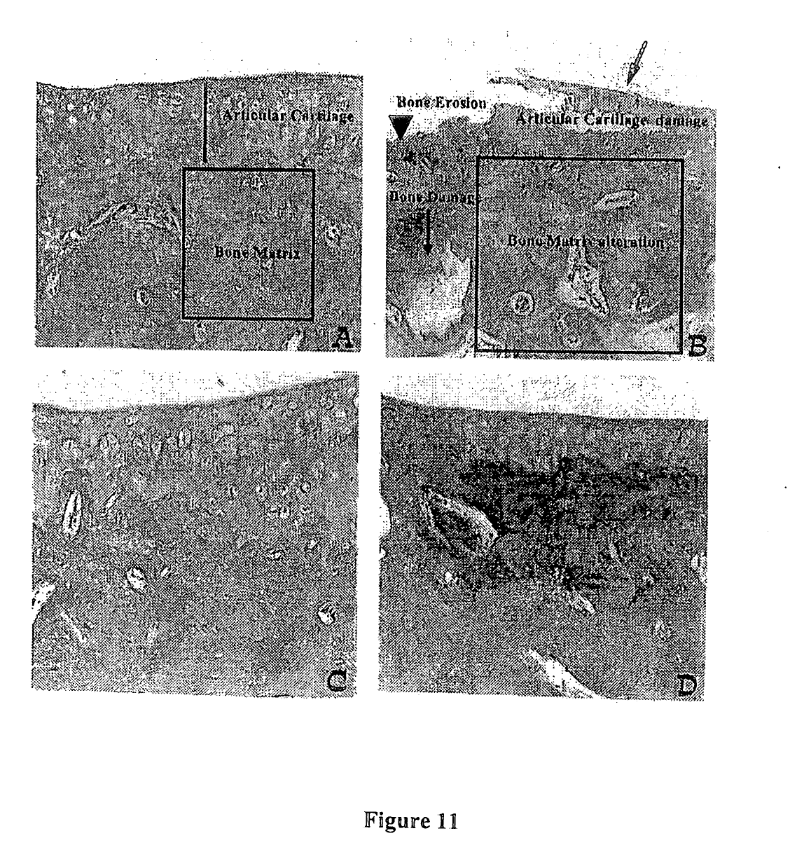

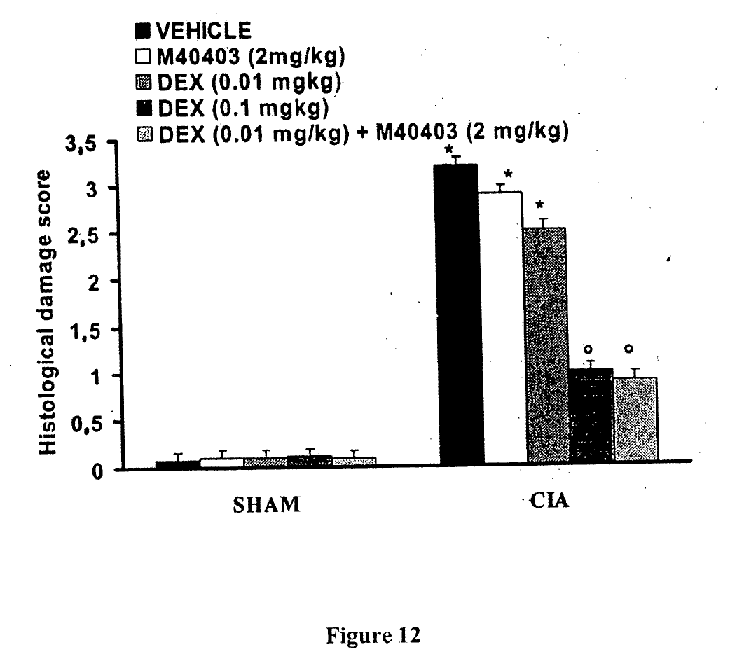

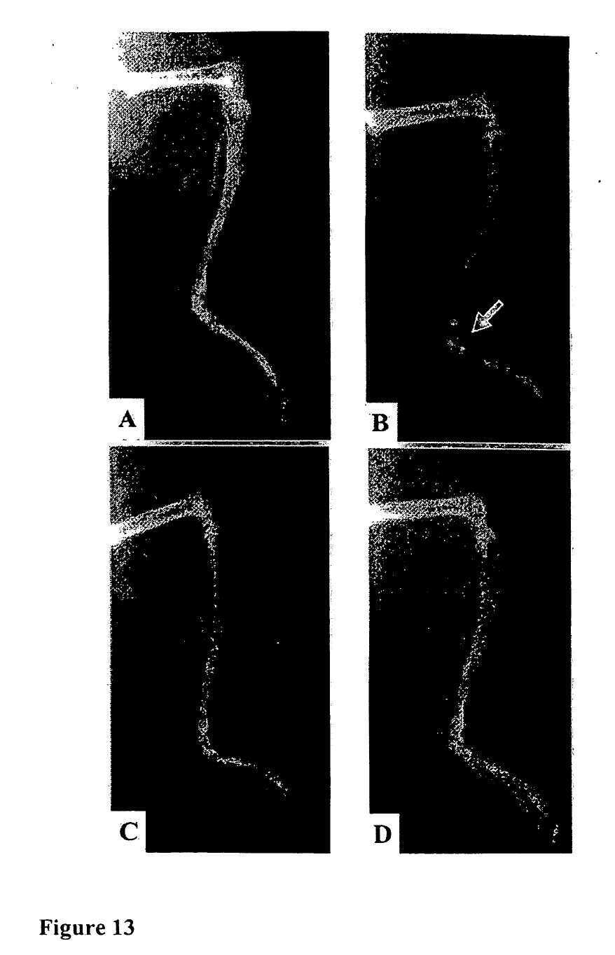

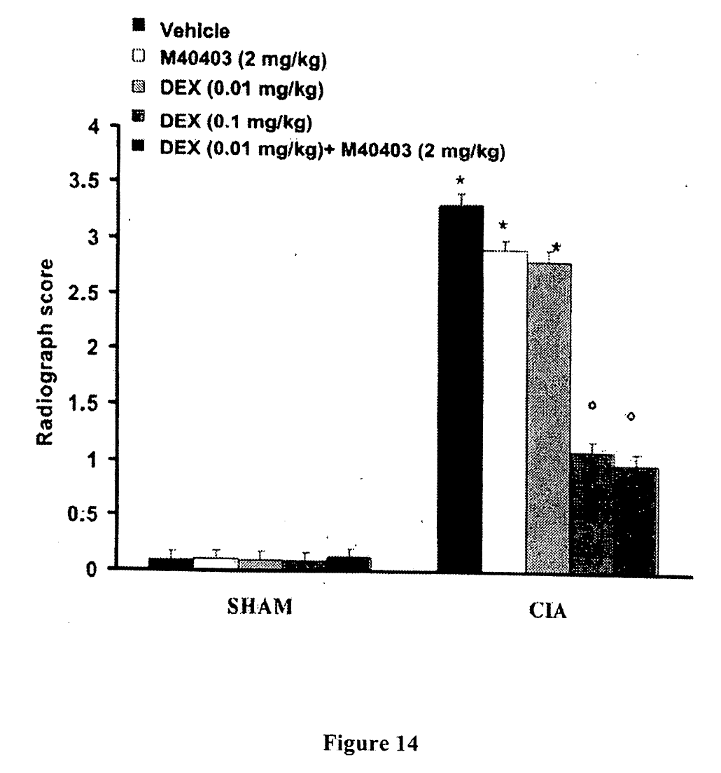

Effects of combination therapy on CIA histopathology and radiographic analysis of CIA. On day 35, histologic evaluation of the knee joint in the vehicle-treated arthritic animals revealed signs of severe suppurative arthritis, with bone resorption (FIG. 11B). In addition, severe or moderate necrosis, hyperplasia, and sloughing of the synovium could be seen, together with extension of the inflammation into the adjacent musculature and fibrosis and increased mucous production (FIG. 12). In the animals that received DEX (0.1 mg/kg) (FIG. 11C) or the combination therapy (M40403 2 mg/kg+DEX 0.01 mg/kg) (FIG. 11D), bone erosion and the degree of arthritis were significantly reduced (FIG. 12). A radiographic examination of rats' hind paws 35 days post-CII immunization revealed bone matrix resorption (FIG. 13B), osteophyte formation at the joint margin, and soft tissue swelling in the tibiotarsal joint (FIG. 14). There was no evidence of pathology in sham-treated rats (FIGS. 11A and 13A). In the proximal tibia, the ObS/Bs, the ES/Bs, and the OcS/Bs were significantly increased 35 days post—CII immunization (Table 2). DEX (0.1 mg/kg) (FIGS. 13C and 14) or the combination therapy (M40403 2 mg/kg+DEX 0.01 mg/kg) (FIGS. 13D and 14 and Table 2) markedly protected animals from bone resorption. No significant protection was found in the animal treated with M40403 (2 mg/kg) or with DEX (0.01 mg/kg) (FIG. 14).

TABLE 2
Histomorphometric findings in the proximal tibia*

| Rat Group | ObS/Bs, % | ES/Bs, % | OcS/Bs, % |
|---|---|---|---|
| Sham + vehicle | 1.18 ± 1.12 | 22.43 ± 2.52 | 1.45 ± 1.02 |
| CII + vehicle | 8 ± 1.12 | 37.74 ± 3.22 † | 2.74 ± 1.2 ‡ |
| CII + M40403 | 2.2 ± 1.04 ‡ | 26.31 ± 2.5 ‡ | 2.74 ± 1.2 ‡ |
| (2 mg/kg) + DEX | | | |
| (0.01 mg/kg) | | | |

*Values are the mean ± SEM. ObS/Bs = osteoblast surface relative to bone surface; ES/Bs = eroded surface relative to bone surface; DEX = dexamethasone.
† P < 0.01 versus sham + vehicle group.
‡ P < 0.01 versus type II collagen (CII) + vehicle group.

### EXAMPLE 3

This example illustrates the biological effect of the use of deacetylated products of dexamethasone and cortisol, reacted with reactive oxygen species

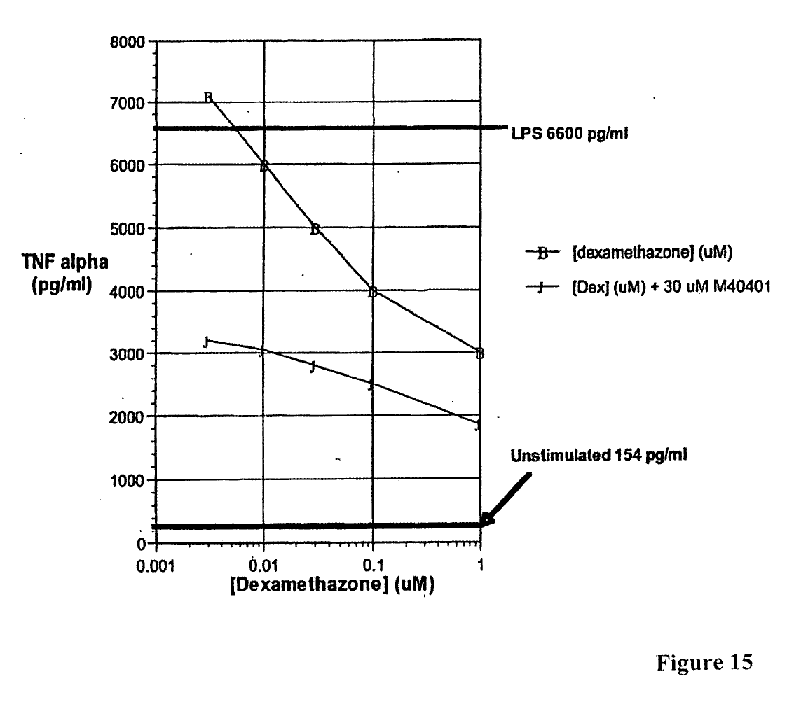

Two compounds were selected as model glucorticoids (dexamethasone and cortisol) for initial study. These were reacted with excess potassium superoxide in protic solvent to yield, upon purification, their respective C-17 deacetylated products. The biological effect of these products was then examined in vitro using the RAW macrophage cell line and whole blood assays. RAW cells are known to respond to LPS with an induction of iNOS and COX-2, as well as with a profound release of TNF-α release. Indeed, dexamethasone causes a dose-dependent inhibition of LPS-stimulated TNF-α as shown in FIG. 15.

FIG. 15 shows that administration of an antioxidant, the SOD mimic designated M40401, to LPS treated RAW cells enhances the effect of dexamethasone.

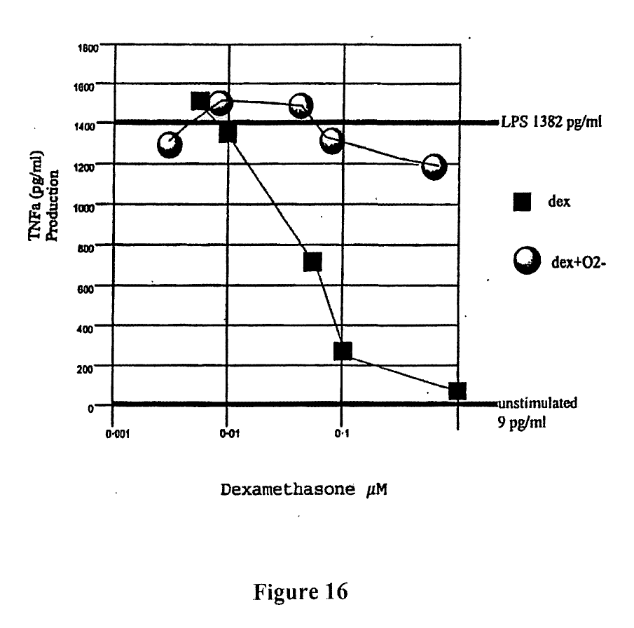

Remarkably, the presence of a superoxide dismutase mimic (M40401), at concentrations sufficiently below its own ability to inhibit the cytokine, causes a profound enhancement in the ability of dexamethasone to inhibit TNF-α. This suggests that LPS-activated macrophages release significant quantities of free radicals (e.g., superoxide and nitric oxide) which, in turn, affect the ability of dexamethasone to exert anti-inflammatory effects. Therefore, glucocorticoids that have been inactivated by free radicals would not be expected to depress nitric oxide, prostaglandin, or TNF-A production in in vitro or in vivo assays. In fact, the oxidation product obtained from the reaction of dexamethasone with superoxide, tested in vitro for its ability to inhibit TNF-α production, was found to have no activity as shown in FIG. 16.

Over the last few decades, there has been significant effort and accomplishment in the area of non-steroidal anti-inflammatory drugs (NSAIDs). NSAIDs (including nitric oxide synthase inhibitors and TNF-antibodies) exert their anti-inflammatory effects farther down the inflammation cascade than do glucocorticoids and they typically have fewer or less severe side effects. However, glucocorticoids were previously known to be the most potent anti-inflammatory agents. If a glucocorticoid could be produced having similar anti-inflammatory properties as dexamethasone while possessing diminished side effects, then inflammation could be mediated at the source of the cascade. The use of a combination therapy of SOD mimics and corticosteroids provides the efficient therapy of corticosteroids with diminished side effects because lower doses of corticosteroids in inflammation may be used.

### EXAMPLE 4

This example illustrates the effects of dexamethasone and FeTMPS in carrageenan-induced paw edema

Methods

Dexamethasone was given by lavage one hour before carrageenan. FeTMPS (1 mg/kg) was given intravenously 15 minutes before carrageenan. Male sprague dawley rats weighing between 200 and 210 g were used. Paw edema was monitored for 6 hours. Results express delta change from basal. Each number is the mean+s.e.m. for n=4 rats per group.

Results

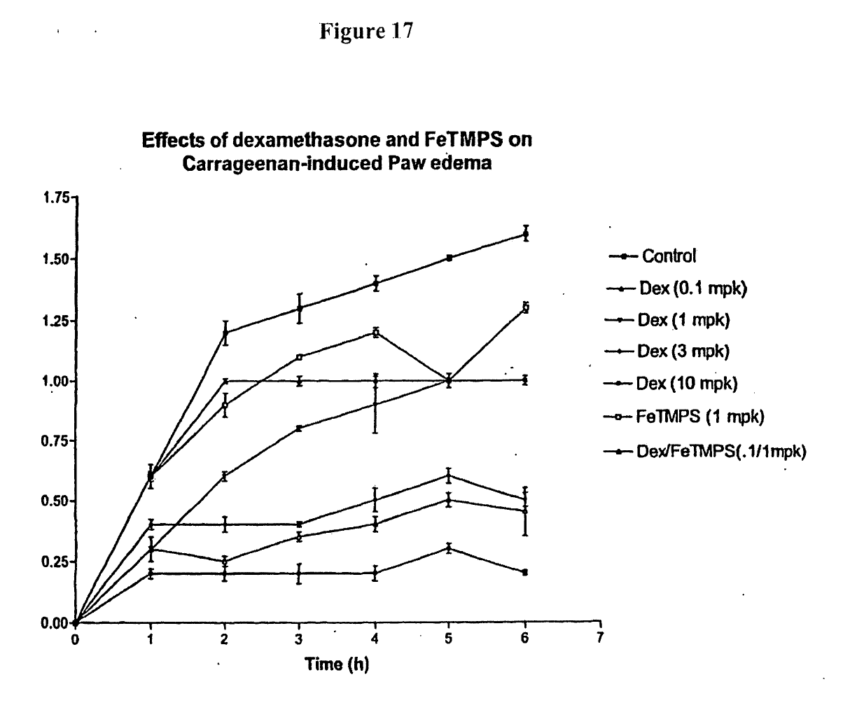

As can be seen from FIG. 17 and the Table 3 below, a low dose of FeTMPS (1 mg/kg) when combined with low dose dexamethasone (0.1 mg/kg) enhances the effects of dexamethasone. The combination of the compound and the low dose of dexamethasone (0.1 mg/kg) is as effective as a 3 mg/kg dose of dexamethasone.

TABLE 3
FeTMPS Effect with Dexamethasone

| | | | | | | | Dex |
|---|---|---|---|---|---|---|---|
| Time (h) | | | | | | | 0.1 mpk ± |
| post | | Dex | Dex | Dex | Dex | FeTMPS | FeTMPS |
| carrageenan | Control | 0.1 mpk | 1 mpk | 3 mpk | 10 mpk | 1 mpk | 1 mk |
| 0 | 0 | 0 | 0 | 0 | 0 | 0 | 0 |
| 1 | 0.6 ± 0.01 | 0.3 ± 0.05 | 0.3 ± 0.05 | 0.4 ± 0.02 | 0.2 ± 0.02 | 0.6 ± 0.02 | 0.3 ± 0.01 |
| 2 | 1.2 ± 0.05 | 1 ± 0.01 | 0.6 ± 0.02 | 0.4 ± 0.03 | 0.2 ± 0.03 | 0.9 ± 0.05 | 0.25 ± 0.02 |
| 3 | 1.3 ± 0.06 | 1 ± 0.02 | 0.8 ± 0.01 | 0.4 ± 0.01 | 0.2 ± 0.04 | 1.1 ± 0.01 | 0.35 ± 0.02 |
| 4 | 1.4 ± 0.03 | 1 ± 0.03 | 0.9 ± 0.012 | 0.5 ± 0.05 | 0.2 ± 0.03 | 1.2 ± 0.02 | 0.4 ± 0.03 |
| 5 | 1.5 ± 0.01 | 1 ± 0.01 | 1 ± 0.01 | 0.6 ± 0.03 | 0.3 ± 0.02 | 1 ± 0.03 | 0.5 ± 0.03 |
| 6 | 1.6 ± 0.03 | 1 ± 0.02 | 1 ± 0.02 | 0.5 ± 0.03 | 0.2 ± 0.01 | 1.3 ± 0.02 | 0.45 ± 0.1 |

In view of the above, it will be seen that the several objectives of the invention are achieved and other advantageous results attained.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A method of treating an inflammatory disease of a bone and/or joint, the method comprising administering to a subject in need thereof, an effective amount of a combination of a non-proteinaceous catalyst for dismutation of superoxide and a corticosteroid.

2. A method of claim 1, wherein amounts of either or both of the catalyst and the corticosteroid in the combination, are less than substantially effective when administered alone, but substantially effective when administered in the combination.

3. A method of claim 1, wherein the disease is selected from the group consisting of rheumatoid arthritis, osteoarthritis, asthma, psoriasis, inflammatory bowel disease, fibromyalgia, systemic lupus erythematosus, scleroderma, juvenile rheumatoid arthritis, ankylosing spondylitis, Sjogren's syndrome, gout, infectious arthritis, reactive arthritis, psoriatic arthritis, bursitis and tendonitis.

4. A method of claim 1, wherein administering an effective amount of the combination improves one or more measures of the inflammatory disease selected form the group consisting of histologic measures, radiographic measures, histomorphometric measures and combinations thereof.

5. A method of claim 4, wherein improved histologic measures comprise preventing or diminishing either or both of bone resorption and infiltration of inflammatory cells.

6. A method of claim 4, wherein the improved radiographic measures comprise preventing or diminishing bone erosion, osteophyte formation, joint erosion or any combination thereof.

7. A method of claim 4, wherein the improved histomorphometric measures comprise either or both of decreasing bone resorption measurements of eroded surface and/or osteoclast surface relative to bone surface, and increasing bone formation measurement of osteoblast surface relative to bone surface.

8. A method of claim 1, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

9. A method of claim 8, wherein the corticosteroid is dexamethasone.

10. A method of claim 9, wherein the dexamethasone is administered parenterally.

11. A method of claim 10, wherein the subject is human and the amount of dexamethasone administered is not more than about 0.0015 mg/kg.

12. A method of claim 1, wherein the non-proteinaceous catalyst is a superoxide dismutase mimetic.

13. A method of claim 12, wherein the superoxide dismutase mimetic is represented by formula: 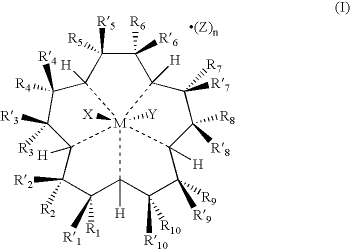
wherein
(i) one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ are independently:
(ia) hydrogen; or
(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino acids, wherein R11 and R12 are independently hydrogen or alkyl; and
(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and
(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰, together with the carbon atom to which they are attached independently form a substituted or unsubstituted and saturated, partially saturated, or unsaturated cycle or heterocycle having 3 to 20 carbon atoms; and
(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R⁵, R′⁵, R⁶, R′⁶, R′⁷, R⁸, R′⁸, R⁹, R′⁹, R¹⁰ and R′¹⁰, which is attached to a different carbon atom in the macrocyclic ligand may be bound to form a strap represented by the formula:
(CH₂)_{I}Q(CH₂)_{J}R(CH₂)_{K}S(CH₂)_{L}
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge neutralizing anions which are derived from any monodentate or polydentate coordinating ligand and a ligand system and the corresponding anion thereof; or
X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸ , R⁹, R′⁹, R¹⁰ and R′¹⁰; and
n is an integer from 0 to 3.

14. A method according to claim 13, wherein the superoxide dismutase mimetic is represented by formula:

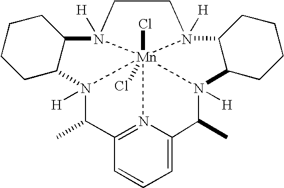

15. A method according to claim 13, wherein the superoxide dismutase mimetic is represented by formula:

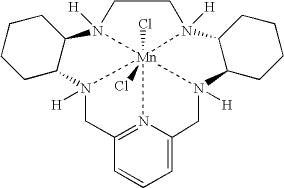

16. A method according to claim 15, wherein the superoxide dismutase mimetic is administered parenterally.

17. A method according to claim 16, wherein the subject is human and the amount of the superoxide dismutase mimetic administered is not more than about 0.25 mg/kg.

18. A method according to claim 1, wherein the catalyst and the corticosteroid are administered in one composition.

19. A method of claim 1, wherein the catalyst and the corticosteroid are administered in separate compositions.

20. A method of effectively treating an inflammatory disease of a bone and/or joint with a reduced dose of a corticosteroid, the method comprising administering to a subject in need thereof, an effective amount of a combination of the corticosteroid and a non-proteinaceous catalyst for dismutation of superoxide, wherein amount of the corticosteroid in the combination, is less than substantially effective when administered alone, but substantially effective when administered in the combination.

21. A method of claim 20, wherein the disease is selected from the group consisting of rheumatoid arthritis, osteoarthritis, asthma, psoriasis, inflammatory bowel disease, fibromyalgia, systemic lupus erythematosus, scleroderma, juvenile rheumatoid arthritis, ankylosing spondylitis, Sjogren's syndrome, gout, infectious arthritis, reactive arthritis, psoriatic arthritis, bursitis and tendonitis, gout, infectious arthritis, reactive arthritis, psoriatic arthritis, bursitis and tendonitis.

22. A method of claim 20, wherein administering an effective amount of the combination improves one or more measures of the inflammatory disease selected form the group consisting of histologic measures, radiographic measures, histomorphometric measures and combinations thereof.

23. A method of claim 22, wherein improved histologic measures comprise preventing or diminishing either or both of bone resorption and infiltration of inflammatory cells.

24. A method of claim 22, wherein the improved radiographic measures comprise preventing or diminishing bone erosion, osteophyte formation, joint erosion or any combination thereof.

25. A method of claim 22, wherein the improved histomorphometric measures comprise either or both of decreasing bone resorption measurements of eroded surface and/or osteoclast surface relative to bone surface, and increasing bone formation measurement of osteoblast surface relative to bone surface.

26. A method of claim 20, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

27. A method of claim 26, wherein the corticosteroid is dexamethasone.

28. A method of claim 27, wherein the dexamethasone is administered parenterally.

29. A method of claim 28, wherein the subject is human and the amount of dexamethasone administered is not more than about 0.0015 mg/kg.

30. A method of claim 20, wherein the catalyst is a superoxide dismutase mimetic.

31. A method of claim 30, wherein the superoxide dismutase mimetic is represented by formula: 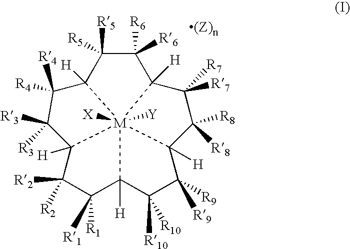
wherein
(i) one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ are independently:
(ia) hydrogen; or
(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino acids, wherein R11 and R12 are independently hydrogen or alkyl; and
(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and
(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰, together with the carbon atom to which they are attached independently form a substituted or unsubstituted and saturated, partially saturated, or unsaturated cycle or heterocycle having 3 to 20 carbon atoms; and
(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, which is attached to a different carbon atom in the macrocyclic ligand may be bound to form a strap represented by the formula:
(CH₂)_{I}Q(CH₂)_{J}R(CH₂)_{K}S(CH₂)_{L}
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge neutralizing anions which are derived from any monodentate or polydentate coordinating ligand and a ligand system and the corresponding anion thereof; or
X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰; and
n is an integer from 0 to 3.

32. A method according to claim 31, wherein the superoxide dismutase mimetic is represented by formula:

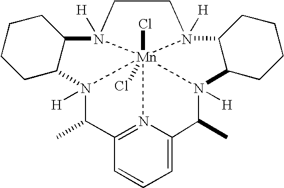

33. A method according to claim 31, wherein the superoxide dismutase mimetic is represented by formula:

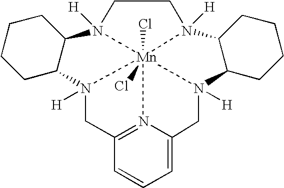

34. A method according to claim 33, wherein the superoxide dismutase mimetic is administered parenterally.

35. A method according to claim 34, wherein the subject is human and the amount of the superoxide dismutase mimetic administered is not more than about 0.25 mg/kg.

36. A method according to claim 20, wherein the catalyst and the corticosteroid are administered in one composition.

37. A method of claim 20, wherein the catalyst and the corticosteroid are administered in separate compositions.

38. A method of increasing the effectiveness of a corticosteroid in treating an inflammatory disease of a bone and/or joint, the method comprising administering to a subject in need thereof, an effective amount of a combination of the corticosteroid and a non-proteinaceous catalyst for dismutation of superoxide, wherein the effectiveness of the combination is greater than that of the corticosteroid when administered alone.

39. A method of claim 38, wherein the disease is selected from the group consisting of rheumatoid arthritis, osteoarthritis, asthma, psoriasis, inflammatory bowel disease, fibromyalgia, systemic lupus erythematosus, scleroderma, juvenile rheumatoid arthritis, ankylosing spondylitis, Sjogren's syndrome, gout, infectious arthritis, reactive arthritis, psoriatic arthritis, bursitis and tendonitis.

40. A method of claim 38, wherein administering an effective amount of the combination improves one or more measures of the inflammatory disease selected form the group consisting of histologic measures, radiographic measures, histomorphometric measures and combinations thereof.

41. A method of claim 40, wherein improved histologic measures comprise preventing or diminishing either or both of bone resorption and infiltration of inflammatory cells.

42. A method of claim 40, wherein the improved radiographic measures comprise preventing or diminishing bone erosion, osteophyte formation, joint erosion or any combination thereof.

43. A method of claim 40, wherein the improved histomorphometric measures comprise either or both of decreasing bone resorption measurements of eroded surface and/or osteoclast surface relative to bone surface, and increasing bone formation measurement of osteoblast surface relative to bone surface.

44. A method of claim 38, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

45. A method of claim 44, wherein the corticosteroid is dexamethasone.

46. A method of claim 45, wherein the dexamethasone is administered parenterally.

47. A method of claim 46, wherein the subject is human and the amount of dexamethasone administered is not more than about 0.0015 mg/kg.

48. A method of claim 38, wherein the catalyst is a superoxide dismutase mimetic.

49. A method of claim 48, wherein the superoxide dismutase mimetic is represented by formula: 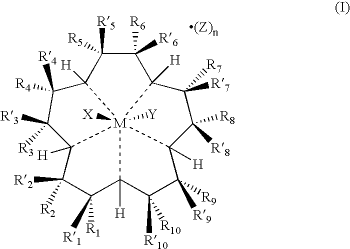
wherein
(i) one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ are independently:
(ia) hydrogen; or
(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino acids, wherein R11 and R12 are independently hydrogen or alkyl; and
(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and
(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰, together with the carbon atom to which they are attached independently form a substituted or unsubstituted and saturated, partially saturated, or unsaturated cycle or heterocycle having 3 to 20 carbon atoms; and
(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R¹, R′, R², R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R′⁵, R⁶, R′⁶, R⁷, R′⁷, R⁸, R′⁸, R⁹, R′⁹, R¹⁰ and R′¹⁰, which is attached to a different carbon atom in the macrocyclic ligand may be bound to form a strap represented by the formula:
(CH₂)_{I}Q(CH₂)_{J}R(CH₂)_{K}S(CH₂)_{L}
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge neutralizing anions which are derived from any monodentate or polydentate coordinating ligand and a ligand system and the corresponding anion thereof; or
X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰; and
n is an integer from 0 to 3.

50. A method according to claim 49, wherein the superoxide dismutase mimetic is represented by formula:

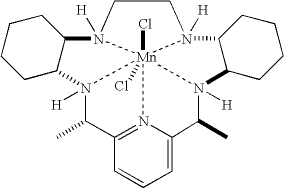

51. A method according to claim 49, wherein the superoxide dismutase mimetic is represented by formula:

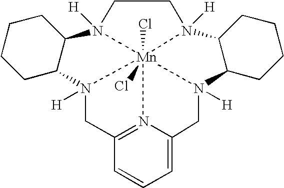

52. A method according to claim 51, wherein the superoxide dismutase mimetic is administered parenterally.

53. A method according to claim 52, wherein the subject is human and the amount of the superoxide dismutase mimetic administered is not more than about 0.25 mg/kg.

54. A method according to claim 38, wherein the catalyst for dismutation of superoxide and the corticosteroid are administered in one composition.

55. A method of claim 38, wherein the catalyst for dismutation of superoxide and the corticosteroid are administered in separate compositions.

56. A method of improving one or more measures of an inflammatory disease selected form the group consisting of histologic measures, radiographic measures, histomorphometric measures and combinations thereof comprising administering to a subject an effective amount of a combination of a non-proteinaceous catalyst for dismutation of superoxide and a corticosteroid.

57. A method of claim 56, wherein amounts of either or both of the catalyst and the corticosteroid in the combination, are less than substantially effective when administered alone, but substantially effective when administered in the combination.

58. A method of claim 56, wherein the disease is selected from the group consisting of rheumatoid arthritis, osteoarthritis, asthma, psoriasis, inflammatory bowel disease, fibromyalgia, systemic lupus erythematosus, scleroderma, juvenile rheumatoid arthritis, ankylosing spondylitis, Sjogren's syndrome, gout, infectious arthritis, reactive arthritis, psoriatic arthritis, bursitis and tendonitis.

59. A method of claim 56, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

60. A method of claim 59, wherein the corticosteroid is dexamethasone.

61. A method of claim 60, wherein the dexamethasone is administered parenterally.

62. A method of claim 61, wherein the subject is human and the amount of dexamethasone administered is not more than about 0.0015 mg/kg.

63. A method of claim 56, wherein the non-proteinaceous catalyst is a superoxide dismutase mimetic.

64. A method of claim 63, wherein the superoxide dismutase mimetic is represented by formula: 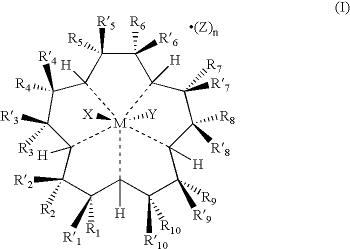
wherein
(i) one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ are independently:
(ia) hydrogen; or
(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino R11 and R12 are independently hydrogen or alkyl; and
(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and
(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰, together with the carbon atom to which they are attached independently form a substituted or unsubstituted and saturated, partially saturated, or unsaturated cycle or heterocycle having 3 to 20 carbon atoms; and
(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R⁵, R′⁵, R⁶, R′⁶, R⁷, ′⁷R, R⁸, R′⁸, R⁹, R′⁹, R¹⁰ and R′¹⁰, which is attached to a different carbon atom in the macrocyclic ligand may be bound to form a strap represented by the formula:
(CH₂)_{I}Q(CH₂)_{J}R(CH₂)_{K}S(CH₂)_{L}
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge neutralizing anions which are derived from any monodentate or polydentate coordinating ligand and a ligand system and the corresponding anion thereof; or
X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰; and
n is an integer from 0 to 3.

65. A method according to claim 64, wherein the superoxide dismutase mimetic is represented by formula:

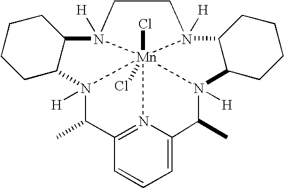

66. A method according to claim 64, wherein the superoxide dismutase mimetic is represented by formula:

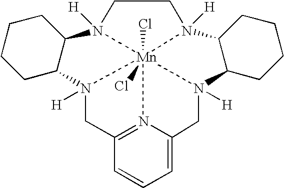

67. A method according to claim 66, wherein the superoxide dismutase mimetic is administered parenterally.

68. A method according to claim 67, wherein the subject is human and the amount of the superoxide dismutase mimetic administered is not more than about 0.25 mg/kg.

69. A method according to claim 56, wherein the catalyst and the corticosteroid are administered in one composition.

70. A method of claim 56, wherein the catalyst and the corticosteroid are administered in separate compositions.

71. A method of preventing or diminishing either or both of bone resorption and infiltration of inflammatory cells comprising administering to a subject an effective amount of a combination of a non-proteinaceous catalyst for dismutation of superoxide and a corticosteroid.

72. A method of claim 71, wherein amounts of either or both of the catalyst and the corticosteroid in the combination, are less than substantially effective when administered alone, but substantially effective when administered in the combination.

73. A method of claim 71, wherein the disease is selected from the group consisting of rheumatoid arthritis, osteoarthritis, asthma, psoriasis, inflammatory bowel disease, fibromyalgia, systemic lupus erythematosus, scleroderma, juvenile rheumatoid arthritis, ankylosing spondylitis, Sjogren's syndrome, gout, infectious arthritis, reactive arthritis, psoriatic arthritis, bursitis and tendonitis.

74. A method of claim 71, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

75. A method of claim 74, wherein the corticosteroid is dexamethasone.

76. A method of claim 75, wherein the dexamethasone is administered parenterally.

77. A method of claim 76, wherein the subject is human and the amount of dexamethasone administered is not more than about 0.0015 mg/kg.

78. A method of claim 71, wherein the non-proteinaceous catalyst is a superoxide dismutase mimetic.

79. A method of claim 78, wherein the superoxide dismutase mimetic is represented by formula: 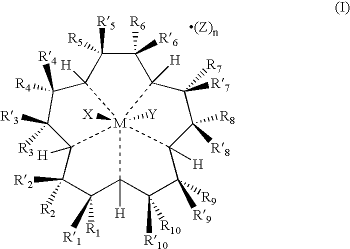
wherein
(i) one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ are independently:
(ia) hydrogen; or
(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino acids, wherein R11 and R12 are independently hydrogen or alkyl; and
(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and
(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰, together with the carbon atom to which they are attached independently form a substituted or unsubstituted and saturated, partially saturated, or unsaturated cycle or heterocycle having 3 to 20 carbon atoms; and
(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, which is attached to a different carbon atom in the macrocyclic ligand may be bound to form a strap represented by the formula:
(CH₂)_{I}Q(CH₂)_{J}R(CH₂)_{K}S(CH₂)_{L}
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge neutralizing anions which are derived from any monodentate or polydentate coordinating ligand and a ligand system and the corresponding anion thereof; or
X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰; and
n is an integer from 0 to 3.

80. A method according to claim 79, wherein the superoxide dismutase mimetic is represented by formula:

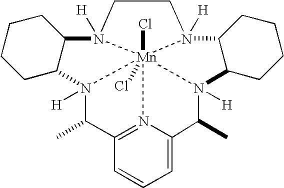

81. A method according to claim 79, wherein the superoxide dismutase mimetic is represented by formula:

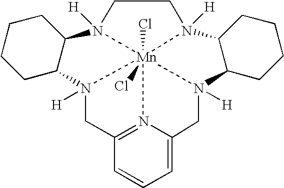

82. A method according to claim 81, wherein the superoxide dismutase mimetic is administered parenterally.

83. A method according to claim 82, wherein the subject is human and the amount of the superoxide dismutase mimetic administered is not more than about 0.25 mg/kg.

84. A method according to claim 71, wherein the catalyst and the corticosteroid are administered in one composition.

85. A method of claim 71, wherein the catalyst and the corticosteroid are administered in separate compositions.

86. A method of preventing or diminishing bone erosion, osteophyte formation, joint erosion or any combination thereof comprising administering to a subject an effective amount of a combination of a non-proteinaceous catalyst for dismutation of superoxide and a corticosteroid.

87. A method of claim 86, wherein amounts of either or both of the catalyst and the corticosteroid in the combination, are less than substantially effective when administered alone, but substantially effective when administered in the combination.

88. A method of claim 86, wherein the disease is selected from the group consisting of rheumatoid arthritis, osteoarthritis, asthma, psoriasis, inflammatory bowel disease, fibromyalgia, systemic lupus erythematosus, scleroderma, juvenile rheumatoid arthritis, ankylosing spondylitis, Sjogren's syndrome, gout, infectious arthritis, reactive arthritis, psoriatic arthritis, bursitis and tendonitis.

89. A method of claim 86, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

90. A method of claim 89, wherein the corticosteroid is dexamethasone.

91. A method of claim 90, wherein the dexamethasone is administered parenterally.

92. A method of claim 91, wherein the subject is human and the amount of dexamethasone administered is not more than about 0.0015 mg/kg.

93. A method of claim 86, wherein the non-proteinaceous catalyst is a superoxide dismutase mimetic.

94. A method of claim 93, wherein the superoxide dismutase mimetic is represented by formula: 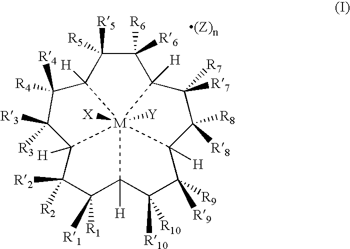
wherein
(i) one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰ are independently:
(ia) hydrogen; or
(ib) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ic) a moiety independently selected from the group consisting of OR¹¹, NR¹¹R¹², COR¹¹, CO₂R¹¹, CONR¹¹R¹², SR¹¹, SOR¹¹, SO₂R¹¹, SO₂NR¹¹R¹², N(OR¹¹)(R¹²), P(O)(OR¹¹)(OR¹²), P(O)(OR¹¹)(R¹²), OP(O)(OR¹¹)(OR¹²), and substituents attached to the α carbon of α amino acids, wherein R11 and R12 are independently hydrogen or alkyl; and
(ii) optionally, one or more of R¹ or R′¹ and R² or R′², R³ or R′³ and R⁴ or R′⁴, R⁵ or R′⁵ and R⁶ or R′⁶, R⁷ or R′⁷ and R⁸ or R′⁸, R⁹ or R′⁹ and R¹⁰ or R′¹⁰ together with the carbon atoms to which they are attached independently form a substituted or unsubstituted and saturated, partially saturated, or unsaturated cycle or heterocycle having 3 to 20 carbon atoms; and
(iii) optionally, one or more of R¹ and R′¹, R² and R′², R³ and R′³, R⁴ and R′⁴, R⁵ and R′⁵, R⁶ and R′⁶, R⁷ and R′⁷, R⁸ and R′⁸, R⁹ and R′⁹, and R¹⁰ and R′¹⁰, together with the carbon atom to which they are attached independently form a substituted or unsubstituted and saturated, partially saturated, or unsaturated cycle or heterocycle having 3 to 20 carbon atoms; and
(iv) optionally, one or more of R¹⁰ or R′¹⁰ and R¹ or R′¹, R² or R′² and R³ or R′³, R⁴ or R′⁴ and R⁵ or R′⁵, R⁶ or R′⁶ and R⁷ or R′⁷, or R⁸ or R′⁸ and R⁹ or R′⁹ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, together with a different one of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰, which is attached to a different carbon atom in the macrocyclic ligand may be bound to form a strap represented by the formula:
(CH₂)_{I}Q(CH₂)_{J}R(CH₂)_{K}S (CH₂)_{L}
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge neutralizing anions which are derived from any monodentate or polydentate coordinating ligand and a ligand system and the corresponding anion thereof; or
X, Y and Z are independently attached to one or more of R¹, R′, R², R′², R³, R′³, R⁴, R′⁴, R⁵, R′⁵, R⁶, R′⁶, R⁷, R′⁷, R⁸, R′⁸, R⁹, R′⁹, R¹⁰ and R′¹⁰; and
n is an integer from 0 to 3.

95. A method according to claim 94, wherein the superoxide dismutase mimetic is represented by formula:

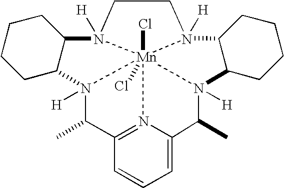

96. A method according to claim 94, wherein the superoxide dismutase mimetic is represented by formula:

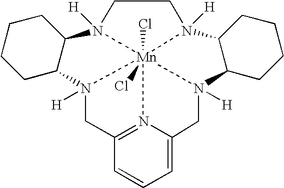

97. A method according to claim 96, wherein the superoxide dismutase mimetic is administered parenterally.

98. A method according to claim 97, wherein the subject is human and the amount of the superoxide dismutase mimetic administered is not more than about 0.25 mg/kg.

99. A method according to claim 86, wherein the catalyst and the corticosteroid are administered in one composition.

100. A method of claim 86, wherein the catalyst and the corticosteroid are administered in separate compositions.
